(19) United States
(12) Patent Application Publication
(10) Pub. No.: US 2023/0263766 A1
(43) Pub. Date: Aug. 24, 2023

(54) CANNABINOID-CONTAINING FORMULATIONS FOR PARKINSONIAN MOVEMENT DISORDERS

(71) Applicant: GBS Global BioPharma, Inc., Ottawa (CA)

(72) Inventor: Andrea Lee Small-Howard, Norwalk, CA (US)

(21) Appl. No.: 17/955,425

(22) Filed: Sep. 28, 2022

Related U.S. Application Data
(60) Provisional application No. 63/249,482, filed on Sep. 28, 2021.
(60) Provisional application No. 63/290,933, filed on Dec. 17, 2021.
(60) Provisional application No. 63/374,582, filed on Sep. 5, 2022.

(51) Int. Cl. A61K 31/352; A61K 31/05; A61P 25/16

(52) U.S. Cl. CPC A61K 31/352; A61K 31/05; A61P 25/16

(57) ABSTRACT

Provided herein are cannabinoid-ratio defined formulations suitable for use as an active pharmaceutical ingredient. Also provided are methods of making the cannabinoid-ratio defined formulations; pharmaceutical compositions comprising the cannabinoid-ratio defined formulations, and methods of using the pharmaceutical compositions for the treatment of Parkinsonian movement disorders, including Parkinson’s disease, parkinsonism, and dopamine-related Parkinson’s disease symptoms.

### 1. CROSS-REFERENCE TO RELATED APPLICATIONS

This application claims the benefit of U.S. Provisional Application Nos. 63/249,482, filed Sep. 28, 2021, and 63/290,933, filed Dec. 17, 2022, and 63/374,582, filed Sep. 5, 2022, each of which applications is hereby incorporated in its entirety.

### 2. BACKGROUND

Neurodegeneration in Parkinson’s disease (PD), Alzheimer’s disease, Lewy Body Dementia, and Huntington’s disease is a growing health burden. Among these, the pathophysiology of PD has been intensively studied, but its underlying cause remains enigmatic. Mechanistically, motor symptoms of PD are linked to the death of dopamine (DA)-producing neurons in the substantia nigra and to the deposition of misfolded alpha-synuclein protein aggregates in Lewy bodies. Desensitization of the DA response system has also been documented, suggesting that both DA production and efficacy are compromised in PD. Most of the agents currently approved for treating PD address symptoms of DA depletion, such as bradykinesia, and do not modify disease progression. Levodopa remains the most common symptomatic treatment for PD; however, 30-35% of patients develop Levodopa Induced Dyskinesia (LID) after as little as 24 months of Levodopa usage. Given these significant side effects, there remains a need for non-Levodopa based symptomatic therapies for PD.

The potential for Cannabis-derived compounds to provide symptom improvement in PD patients is suggested by anecdotal and patient reported outcome (PRO) data. Unfortunately, with native Cannabis or Cannabis extracts, there are unnecessary and unwanted psychoactive side effects from delta-9 tetrahydrocannabinol (THC), along with plant material contamination and complexities in the accurate delivery of therapeutic extracts that may compromise patient safety.

Additionally, single cannabinoid therapeutics composed of THC or cannabidiol (CBD) do not fully recapitulate the PRO effectiveness of the native plant or it’s extracts. This suggests that Cannabis plant extracts, which contain hundreds of compounds, include components other than these major cannabinoids that contribute significantly to their effectiveness. The pharmacodynamic properties of the cannabinoid and terpene active ingredients from Cannabis plant extracts have been described in detail. In the cannabinoid research field, the ability of Cannabis-derived ingredients to act synergistically by enhancing or diminishing the net effectiveness of a therapy has been identified and is referred to as the ‘entourage effect’. While the entourage effect is typically expected to be modulated pharmacodynamically through the interactions of multiple ligands with one or more receptors, pharmacokinetic effects such as metabolism have also been demonstrated. This makes assessing their activity more complicated, but also makes them potentially more effective therapeutics than single target drugs due to positive co-operative interactions. The cannabinoids and terpenes from Cannabis plant extracts are ligands of multiple receptors including metabotropic cannabinoid receptors, ionotropic cannabinoid receptors, serotonin receptors, and orphan G-protein coupled receptors, making it likely that they would individually act as multi-target drugs, and indeed Cannabis extracts demonstrate more potency than CBD alone in cell based assays. A number of minor cannabinoids have been shown to bind to CB1 and CB2 receptors, although with varying affinities. While some terpenes have shown an additive effect with cannabinoid agonists in rodents, the direct interaction of terpenes with the CB receptors and TRP channels is contested. Developing a model to study these complex interactions is a critical step to the rational design of multi-component, efficacious, Cannabis-inspired therapeutics for PD.

Based on the complexity, side effects, and off target interactions that may be inevitable using whole plant extracts, it is essential to identify the core components of Cannabis that are required for the treatment of PD. Using a combination of in silico and cell based assays, the inventors of the present disclosure previously identified a mixture of essential cannabis components that when combined with CBD or cannabinol (CBN), recapitulated the anti-Parkinsonian activity anecdotally ascribed to whole plant Cannabis. These compounds include the three minor cannabinoids cannabigerol (CBG), cannabichromene (CBC) and Cannabidivarin (CBDV) (<5% of the original cannabis extracts), and five terpenes (alpha-pinene, trans-nerolidol, limonene, linalool and phytol). While the identification of 8 compounds from a pool of >100 000 represents a tremendous reduction in complexity, unfortunately these mixtures remain difficult to formulate into therapeutics owing to the diversity of the chemical structures and the differences in pharmacokinetics of each component. FDCs can pose a challenge with dosage adjustments of individual drugs, drug interactions, and off-target effects, with each additional ingredient creating more opportunities for adverse reactions. Thus, the identification of the minimal set of cannabis ingredients that can recapitulate the effects of whole plant is crucial in the creation of a multicomponent therapeutic.

Therefore, the inventors of the present disclosure sought to further reduce the number of compounds in the previously tested formulations to a minimal essential mixture (MEM) that could recapitulate as many of the effects of the original combination as possible with the goal of generating a mixture that would be more amenable to pharmacological production. Two cell assays were initially used to evaluate the potential therapeutic efficacy of the mixtures by using both an in vitro neuroprotection assay and a dopamine secretion assay in dopaminergic neuronal cell models. From these cell assays, the cannabinoids were identified as being largely responsible for the activity seen in the previously tested mixture with a nominal effect of the terpenes.

Multiple drug combinations were then assayed that contained three individual cannabinoids for their ability to ameliorate a 6-hydroxydopmamine (OHDA)-induced model of PD in zebrafish larvae. The results provided moderately minimal essential mixtures (MEMs) with potential PD-therapeutic activity as evaluated in cell models, to refined minimal essential mixtures of cannabinoids that demonstrate therapeutic effects on OHDA treated zebrafish. The sequentially reductionist process used in this study preserves some of the entourage-like effects of whole plant extracts, while achieving ‘relative’ simplicity within MEM that is a requirement for obtaining the manufacturing and quality control advantages of single ingredient drugs.

Medicinal Cannabis has shown promise for the symptomatic treatment of Parkinson’s disease (PD), but patient exposure to whole plant mixtures may be undesirable due to concerns around safety, consistency, regulatory issues, and psychoactivity. Identification of a subset of components responsible for the potential therapeutic effects within Cannabis represents a direct path forward for the generation of anti-PD drugs. The present inventors utilized a reductionist approach to identify a minimal essential mixture (MEM) of these cannabinoids that are amenable to pharmacological formulation. In the first phase, cell-based models revealed that the cannabinoids had the most significant positive effects on neuroprotection and dopamine secretion. Then, the ability of combinations of these cannabinoids was evaluated to ameliorate a 6-hydroxydopmamine (OHDA)-induced change in locomotion in larval zebrafish, which has become a well-established PD disease model. Equimolar mixtures that each contained three cannabinoids were able to significantly reverse the OHDA mediated changes in locomotion and other advanced metrics of behavior. Additional screening of sixty-three variations of the original cannabinoid mixtures identified five highly efficacious mixtures that outperformed the original equimolar cannabinoid MEMs and represent the most attractive candidates for therapeutic development. This work highlights the strength of the reductionist approach for the development of ratio-controlled, Cannabis mixture-based therapeutics for the treatment of Parkinson’s disease.

### 3. SUMMARY

The present disclosure provides new cannabinoid-ratio defined formulations suitable for use as active pharmaceutical ingredients, methods of making the minimal essential mixtures (MEMs), and pharmaceutical compositions comprising the minimal essential mixtures (MEMs).

The present disclosure is based in part on the discovery that cannabinoid mixtures (e.g., various ratios of the OTMs described herein) were able to significantly reverse the OHDA mediated changes in locomotion and other advanced metrics of behavior associated with Parkinson’s disease. The potential of each minimum essential mixture (MEM) of cannabinoids to ameliorate a 6-hydroxydopmamine (OHDA)-induced decrease in locomotion in larval zebrafish was assessed. Exposure to OHDA leads to a depletion of dopaminergic neurons in larval zebrafish and results in decreased locomotive behavior and a ‘resting tremor′-like phenotype. Three equimolar cannabinoid mixtures were able to significantly reverse the OHDA mediated changes in locomotion and other advanced metrics of behavior. Additional screening of sixty-three variations of the original three equimolar cannabinoid mixtures identified a total of twenty-two MEMs with optimized ratios of cannabinoids that produced a statistically significant reduction in OHDA induced motor symptoms. Five of these twenty-two efficacious MEMs outperformed the original equimolar cannabinoid MEMs. This work highlights the potential for development of cannabinoid mixture-based therapeutics for the treatment of Parkinson’s disease.

An aspect of the present disclosure provides a pharmaceutically active ingredient, comprising: a 1:2:10 ratio of: (i) cannabidiol (CBD), (ii) cannabidivarin (CBDV), and (iii) cannabigerol (CBG). In some embodiments, the pharmaceutically active ingredient is in a pharmaceutical composition and also includes a pharmaceutically acceptable carrier or diluent.

In another aspect, the present disclosure provides a pharmaceutically active ingredient comprising a 1:1:1; 2:1:2; 2:1:1; 1:2:2; 1:2:1; 1:10:10; 1:5:10; or 1:1:10 ratio of: (i) cannabidiol (CBD), (ii) cannabidivarin (CBDV), and (iii) cannabigerol (CBG). The pharmaceutically active ingredient can be included in a pharmaceutical composition comprising the pharmaceutically active ingredient and a pharmaceutically acceptable carrier or diluent.

In another aspect, the present application provides a pharmaceutically active ingredient, comprising: a ratio of 2:1:2 of: (i) cannabidiol (CBD), (ii) cannabidivarin (CBDV), and (iii) cannabichromene (CBC). The pharmaceutically active ingredient can be included in a pharmaceutical composition comprising the pharmaceutically active ingredient and a pharmaceutically acceptable carrier or diluent.

An aspect of the present disclosure includes a pharmaceutically active ingredient, comprising: a ratio of: (a) cannabidiol (CBD), (b) cannabidivarin (CBDV), and (c) cannabichromene (CBC).

In some embodiments, the ratio is a molar ratio. In some embodiments, the ratio is selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of: (a) cannabidiol (CBD), (b) cannabidivarin (CBDV), and (c) cannabichromene (CBC). In some embodiments, the ratio is 2:1:2 of CBD, CBDV, and CBC.

Aspects of the present disclosure include a pharmaceutical composition comprising the pharmaceutically active ingredient of the present disclosure and a pharmaceutically acceptable carrier or diluent. In some embodiments, the active ingredient is present in the pharmaceutical composition at a concentration of at least 0.01 mg/ml. In some embodiments, the active ingredient is present in the pharmaceutical composition at a concentration of at least 0.1 mg/ml. In some embodiments, the active ingredient is present in the pharmaceutical composition at a concentration of at least 0.5 mg/ml. In some embodiments, the active ingredient is present in the pharmaceutical composition at a concentration of at least 1 mg/ml, at least 10 mg/ml, or at least 20 mg/ml.

In some embodiments, the composition is formulated for oral administration.

In some embodiments, the pharmaceutical composition is administered as an oral disintegrating tablet (ODT).

Aspects of the present disclosure include a unit dosage form comprising the active pharmaceutical ingredient of the present disclosure.

In some embodiments, the unit dosage form of the active pharmaceutical ingredient for a 2:1:2 ratio of CBD:CBDV:CBC, comprises i. 0.2 mg to 40 mg of CBD; ii. 0.1 mg to 20 mg of CBDV; and iii. 0.2 mg to 40 mg of CBC. In some embodiments, the unit dosage comprises 3 mg of CBD, 1.5 mg of CBDV, and 3 mg of CBC. In some embodiments, the unit dosage comprises 1-5 mg of CBD, 0.05 mg to 3 mg of CBDV, and 1-5 mg of CBC.

An aspect of the present disclosure includes a pharmaceutically active ingredient, comprising: a ratio of: (a) cannabidiol (CBD), (b) cannabichromene (CBC), and (c) cannabidiol (CBN).

The pharmaceutically active ingredient of claim 38, wherein the ratio is a molar ratio.

The pharmaceutically active ingredient of any one of claims 38-39, wherein the ratio is selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of:(a) cannabidiol (CBD), (b) cannabichromene (CBC), and (c) cannabidiol (CBN).

The pharmaceutically active ingredient of claim 40, wherein the ratio is 1:1:1 of CBD, CBC, and CBN.

A pharmaceutical composition comprising the pharmaceutically active ingredient of any one of claims 38-41 and a pharmaceutically acceptable carrier or diluent.

The pharmaceutical composition of claim 42, wherein the active ingredient is present in the pharmaceutical composition at a concentration of at least 0.01 mg/ml.

The pharmaceutical composition of claim 43, wherein the active ingredient is present in the pharmaceutical composition at a concentration of at least 0.1 mg/ml.

The pharmaceutical composition of claim 44, wherein the active ingredient is present in the pharmaceutical composition at a concentration of at least 0.5 mg/ml.

The pharmaceutical composition of claim 45, wherein the active ingredient is present in the pharmaceutical composition at a concentration of at least 1 mg/ml, at least 10 mg/ml, or at least 20 mg/ml.

The pharmaceutical composition of any of the claims 42-46, wherein the composition is formulated for oral administration.

The pharmaceutical composition of any one of claims 42-47, wherein the pharmaceutical composition is administered as an oral disintegrating tablet (ODT).

Another aspect of the present disclosure includes a unit dosage form comprising the active pharmaceutical ingredient of the present disclosure.

In some embodiments, the unit dosage form, for a 1:1:1 ratio of CBD: CBN: CBD, comprises: i. 0.1 mg to 20 mg of CBD; ii. 0.1 mg to 20 mg of CBN; and iii. 0.1 mg to 20 mg of CBC. In some embodiments, the unit dosage comprises 2.5 mg of CBD, 2.5 mg of CBN, and 2.5 mg of CBC. In some embodiments, the unit dosage comprises 0.5 mg -5 mg of CBD, 0.5 mg to 5 mg of CBN, and 0.5 mg to 5 mg of CBC.

Another aspect of the present disclosure includes a pharmaceutically active ingredient, comprising: a ratio of: (a) cannabidiol (CBD), (b) cannabidivarin (CBDV), and (c) cannabigerol (CBG).

In some embodiments, the ratio is a molar ratio. In some embodiemtsn, the ratio is selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of: (a) cannabidiol (CBD), (b) cannabidivarin (CBDV), and (c) cannabigerol (CBG).

In some embodiments, the ratio is 1:2:1 of CBD, CBDV, and CBG.

Aspects of the present disclosure include a pharmaceutical composition comprising the pharmaceutically active ingredient of the present disclosure and a pharmaceutically acceptable carrier or diluent.

In some embodiments, the active ingredient is present in the pharmaceutical composition at a concentration of at least 0.01 mg/ml. In some embodiments, the active ingredient is present in the pharmaceutical composition at a concentration of at least 0.1 mg/ml. In some embodiments, the active ingredient is present in the pharmaceutical composition at a concentration of at least 0.5 mg/ml. In some embodiments, the active ingredient is present in the pharmaceutical composition at a concentration of at least 1 mg/ml, at least 10 mg/ml, or at least 20 mg/ml.

In some embodiments, the composition is formulated for oral administration.

In some embodiments, the pharmaceutical composition is administered as an oral disintegrating tablet (ODT).

Another aspect of the present disclosure includes a unit dosage form comprising the active pharmaceutical ingredient of the present disclosure.

In some embodiments, for a 1:2:1 ratio of CBD:CBDV:CBG, the unit dosage comprises i. 0.1 mg to 20 mg of CBD; ii. 0.2 mg to 40 mg of CBDV; and iii. 0.1 mg to 20 mg of CBG. In some embodiments, the unit dosage comprises 2.5 mg of CBD, 5 mg of CBDV, and 2.5 mg of CBG. In some embodiments, the unit dosage comprises 1 mg to 5 mg of CBD, 2.5 mg to 8 mg of CBDV, and 1 mg to 5 mg of CBG.

In some embodiments, for a 1:2:10 ratio of CBD:CBDV:CBG, the unit dosage comprises: i. 0.01 mg to 5 mg CBD; ii. 0.02 mg to 10 mg CBDV iii. 1.0 mg to 50 mg CBG.

In another aspect, the present application provides a pharmaceutically active ingredient, comprising: a ratio 1:1:1; 1:2:1; 10:10:1; 10:1:1; 1:2:2; or 1:10:10 of: (i) cannabidiol (CBD), (ii) cannabidivarin (CBDV), and (iii) cannabichromene (CBC). The pharmaceutically active ingredient can be included in a pharmaceutical composition comprising the pharmaceutically active ingredient and a pharmaceutically acceptable carrier or diluent.

In another aspect, the present application provides a pharmaceutically active ingredient, comprising: a ratio of 2: 1:1 of: (i) cannabidiol (CBD), (ii) cannabichromene (CBC), and (iii) cannabinol (CBN). The pharmaceutically active ingredient can be included in a pharmaceutical composition comprising the pharmaceutically active ingredient and a pharmaceutically acceptable carrier or diluent.

In another aspect, the present application provides a pharmaceutically active ingredient, comprising: a ratio of 1:1:1; 10:10:1; 10:1:5; 10:1:1; or 1:5:10 of: (i) cannabidiol (CBD), (ii) cannabichromene (CBC), and (iii) cannabinol (CBN). The pharmaceutically active ingredient can be included in a pharmaceutical composition comprising the pharmaceutically active ingredient and a pharmaceutically acceptable carrier or diluent.

Aspects of the present disclosure include methods of treating a Parkinsonian movement disorder, the method comprising: administering an effective amount of any one of the pharmaceutical compositions of the present disclosure to a patient having a Parkinsonian movement disorder.

Another aspect of the present disclosure relates to a method of treating Parkinsonian movement disorder by administering an effective amount of the pharmaceutical composition disclosed herein to a patient having a Parkinsonian movement disorder. In some embodiments, the Parkinsonian movement disorder is Parkinson’s disease.

Another aspect of the present disclosure relates to a method of treating a dopamine-related Parkinson’s Disease symptom by administering an effective amount of the pharmaceutical composition disclosed herein to a patient having a a dopamine-related Parkinson’s Disease symptom. In some embodiments, the dopamine-related Parkinson’s Disease symptom comprises one or more of: tremors, rigidity (e.g., stiffness of the limbs), slowness of movement, and impaired balance and coordination, trembling of the hands, arms, legs, and/or jaw, In some embodiments, the dopamine-related Parkinson’s Disease symptom comprises one or more of: dopamine-related difficulties in concentrating, poor coordination, stooped posture, loss of smell, disruptions in the reward centers of the brain, anxiety, and depression.

In some embodiments, the pharmaceutical composition is administered by inhalation, orally, by buccal administration, by sublingual administration, by injection, or by topical application. In some embodiments, the pharmaceutical composition is administered by orally or by an oral disintegrating tablet (ODT). In some embodiments, the pharmaceutical composition is administered p.r.n.

In some embodiments, the pharmaceutical composition is administered at least once a day. In some embodiments, the pharmaceutical composition is administered at least 2-4 times a day. In some embodiments, the pharmaceutical composition is administered at least 2-4 times a week. In some embodiments, the pharmaceutical composition is administered at least once a week. In some embodiments, the pharmaceutical composition is administered at least once every two weeks.

In one aspect, the present disclosure provides new cannabinoid-ratio defined formulations suitable for use as active pharmaceutical ingredients, methods of making the minimal essential mixtures (MEMs), and pharmaceutical compositions comprising the minimal essential mixtures (MEMs).

In another aspect, the present disclosure provides a method of treating a Parkinsonian movement disorder by administering an effective amount of the pharmaceutical composition disclosed herein to a patient having a Parkinsonian movement disorder. In some embodiments, the Parkinsonian movement disorder is Parkinson’s disease. In another aspect, the present disclosure provides a method of treating a dopamine-related Parkinsonian symptom by administering an effective amount of the pharmaceutical composition disclosed herein to a patient having Parkinson’s Disease with one or more parkinsonian symptoms or dopamine-related Parkinson’s Disease symptoms selected from: Parkinsonian movement disorder, anxiety, depression, reward center dysfunction, poor concentration, loss of smell, loss of coordination, loss of balance, tremors, rigidity (e.g., stiffness of the limbs), slowness of movement, impaired balance and coordination, trembling of the hands, arms, legs, and/or jaw, dopamine-related difficulties in concentrating, poor coordination, stooped posture, loss of smell, disruptions in the reward centers of the brain, and bradykinesia.

Another aspect of the present disclosure includes a method of treating neurodegenerative disease, the method comprising: administering an effective amount of one of the pharmaceutically active ingredients of the present disclosure or a pharmaceutical composition of the present disclosure to a patient having a neurodegenerative disease.

In some embodiments, the neurodegenerative disease is Alzheimer’s disease, Parkinson’s disease, Parkinsonian movement disorder, Lewy Body Dementia, or Huntington’s disease.

In some embodiments, the neurodegenerative disease is Parkinson’s disease or Parkinsonian movement disorder.

In some embodiments, the pharmaceutical composition is administered p.r.n.

In some embodiments, the pharmaceutical composition is administered orally.

In some embodiments, the pharmaceutical composition is administered as an oral disintegrating tablet (ODT).

In some embodiments, the pharmaceutical composition is administered at least once a day.

In some embodiments, the pharmaceutical composition is administered at least 2-4 times a day.

In some embodiments, the pharmaceutical composition is administered at least 2-4 times a week.

In some embodiments, the pharmaceutical composition is administered at least once a week.

In some embodiments, the pharmaceutical composition is administered at least once every two weeks.

Another aspect of the present disclosure includes a method of treating a dopamine-related Parkinson’s Disease symptom, the method comprising: administering an effective amount of one of the pharmaceutically active ingredients of the present disclosure or a pharmaceutical composition of the present disclosure to a patient having a dopamine-related Parkinson’s Disease symptom.

In some embodiments, the dopamine-related Parkinson’s Disease symptom is selected from one or more of: bradykinesia (parkinsonism), slowness with decrement and degradation of repetitive movements (slowness of movement), tremor, rigidity, impaired balance and coordination, stooped posture, loss of smell, disruptions in the reward centers of the brain, anxiety, and depression.

In some embodiments, the dopamine-related Parkinson’s Disease symptom is anxiety.

In some embodiments, the dopamine-related Parkinson’s Disease symptom is depression.

In some embodiments, the patient has Parkinson’s Disease.

In some embodiments, the pharmaceutical composition is administered p.r.n.

In some embodiments, the pharmaceutical composition is administered orally.

In some embodiments, the pharmaceutical composition is administered orally as an oral disintegrating tablet (ODT).

In some embodiments, the pharmaceutical composition is administered at least once a day. In some embodiments, the pharmaceutical composition is administered at least 2-4 times a day.

In some embodiments, the pharmaceutical composition is administered at least 2-4 times a week.

In some embodiments, the pharmaceutical composition is administered at least once a week.

In some embodiments, the pharmaceutical composition is administered at least once every two weeks.

### 4. BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 shows cannabinoids produce significant neuroprotection in MPTP/MPP+ assay. Data are presented as the percent protection from MPTP/MPP cell death evaluated based on the MTT cell viability assay, where the experimental value is normalized relative to the vehicle control. An asterisk * indicates a p-value <0.05 for the replicates relative to their respective vehicle control replicates. MIX-1, MIX-2, and MIX-3 tested without or with the addition of a major cannabinoid (CBD or CBN) a hashtag # represents p < 0.05 major cannabinoid vs native mixture. Calculated (hashed shading) prediction of efficacy based on the sum of the efficacy of each ingredient measured separately and Measured (open shading) efficacy are shown. Each data point in the figure represents the mean +/- the standard deviation of twenty-four experimental results obtained at 10 µM of each major or minor cannabinoid and terpene (alone or in equimolar mixtures as described in the inset table in Panel). Twenty-four experimental results were obtained by repeating eight independent experiments three times on three different days (“8x3”).

FIG. 2 shows cannabinoid mixtures elicit significant dopamine secretion. The experimental value is presented as the normalized value, which is a percent of the positive control value (secretion achieved with PMA/Ionomycin application). An asterisk * indicates a p-value <0.05 for the replicates relative to their respective vehicle control replicates. MIX-1, MIX-2, and MIX-3 were tested without or with the addition of a major cannabinoid (CBD or CBN). A hashtag # represents p < 0.05 major cannabinoid vs native mixture. Calculated (hashed shading) prediction of efficacy based on the sum of the efficacy of each ingredient measured separately and Measured (open shading) efficacy were shown. Each data point in the figure represents the mean +/- the standard deviation of twenty-four experimental results obtained at 10 µM of each major or minor cannabinoid and terpene (alone or in equimolar mixtures as described in the inset table in FIG. 2B). Twenty-four experimental results were obtained by repeating eight independent experiments three times on three different days (“8x3”).

FIGS. 3A-3D shows validation of the larval zebrafish OHDA model. FIG. 3A shows Behavioral profiles of total distance traveled (60 second bins) following OHDA exposure from 48-120 hpf. FIG. 3B shows the total distance travelled during the first 90 minutes in the light following OHDA exposure from 48-120 hpf (n = 36). Advanced activity analytics (FIG. 3C and FIG. 3D) of Total Frequency of switching between activity states (FIG. 3C) and the nested Cumulative Duration in each activity state (FIG. 3D) (n = 48). CC vs OHDA, ** = p < 0.01 **** = p < 0.0001.

FIGS. 4A-4C shows Equimolar Minimum Essential Mixtures (E-MEM) alleviate OHDA mediated hypoactivity. FIG. 4A. Five cannabinoids (CBs) were used to create the 10 possible three component equimolar mixtures. FIGS. 4B-4C. Each of the 500 nM E-MEM (166.7 nM of each cannabinoid) was assessed for its ability modify total distance travelled of B - Carrier Control (CC) or C - 150 µM OHDA. Data is normalized to either CC (FIG. 4B) or OHDA (FIG. 4C) (100%). MEM+OHDA vs OHDA, * = p < 0.05, ** p < 0.01.

FIGS. 5A-5F shows Equimolar MEMs alleviate both OHDA mediated Total Frequency and Cumulative Duration deficits. Activity metrics of Total Frequency (FIG. 5A - FIG. 5B), Cumulative Duration (FIG. 5C - FIG. 5D) and Total Distance (FIG. 5E - FIG. 5F) for equimolar MEMs at 500, 250 and 100 nM (166.7, 83.3 and 33.3 nM each ingredient respectively). MEM vs methanol carrier control (FIG. 5A, FIG. 5C and FIG. 5E), and MEM+150 µM OHDA vs 150 µM OHDA (FIGS. 5B, 5D and 5E). * p < 0.05, ** p < 0.01, *** p < 0.001.

FIGS. 6A-6D shows Optimal DCR-MEM ratios alleviate both OHDA mediated frequency and cumulative duration deficits. Activity metrics of Total Frequency (FIG. 6A - FIG. 6C) and Cumulative Duration (FIG. 6B - FIG. 6D). Optimal DCR-MEM vs methanol carrier control (FIG. 6A and FIG. 6B), and Optimal DCR-MEM+150 µM OHDA vs 150 µM OHDA (FIG. 6C and FIG. 6D). * = p < 0.05, ** = p < 0.01, *** = p < 0.001.

FIGS. 7A-7B show the neuroprotective effects of each individual compound tested at 5 different concentrations. Based on these results, the individual and equimolar mixtures were tested in the cell assays at 10 µM each. Equimolar mixtures contained 10 µM of each compound. In all cases, vehicle controls contained methanol at equal concentrations to those found in test compounds/mixtures, ≤ 5%.

FIG. 8 shows the compound concentration and time exposure paradigm in zebrafish to establish a 6-Hydroxydopamine Parkinson’s model development in zebrafish.

FIG. 9 shows a table (Table 1) containing cannabinoid dilution series testing results. The effects of purified individual cannabinoids was tested acutely on 120 hpf zebrafish larvae. Also, the pure individual cannabinoids were also evaluated for their ability to reverse OHDA-mediated hypoactivity. L/D = Light/dark startle response.

FIGS. 10A-10B provide Table 2 (FIG. 10A) and Table 3 (FIG. 10B) showing a summary of efficacy of defined cannabinoid ratio MEM activities in the OHDA assay in zebrafish. In FIG. 10A, the concentrations of each of the three cannabinoids tested shown in each field of this table are shown starting with the CBD concentration (labeled in the top row). The CBD concentration is used to divide the results table into three columns. The second cannabinoid and concentration for each ratio result is in the first column of the table and is used to further divide the table into nine sets of nine results, and the last cannabinoid and concentration is above the cell in the row containing the label of the original equimolar ratio formula. Astrices (* = p < 0.05, ** = p < 0.01, *** = p < 0.001) on either side of the “/” represent the level of statistical significance in change in the Total Frequency of Activity State Change metric (right-side) / Cumulative Duration metric (left-side) of zebrafish exposed to the MEM + OHDA versus the OHDA-alone group. Zeroes represent no statistically-significant change in activity, “-” indicates a further reduction (increase in PD-like symptoms, p < 0.05)) in activity. N/A = combinations not selected because of the inactivity of their precursors. Cells bold and underlined in italics also showed an MEM dependent increase on methanol treated control larvae. FIG. 10B provides the ratios of each of the MEMs tested.

FIG. 11 provides a table (Table 4) of the chemical, stock concentration and dosage range of the cannabinoids for the acute behavioral assays of Example 1.

### 5. DETAILED DESCRIPTION

### 5.1. Definitions

A pharmaceutically active ingredient is “substantially free” of a compound if the ingredient contains less than 0.3% (w/v) of the compound. For example, a pharmaceutically active ingredient is “substantially free of THC” if the ingredient contains less than 0.3% (w/v) of delta-9 tetrahydrocannabinol. A pharmaceutical composition comprising a pharmaceutically active ingredient is “substantially free of THC” if the pharmaceutical composition contains less than 0.3% (w/v) of delta-9 tetrahydrocannabinol.

A “Cannabis sativa extract” is a composition obtained from Cannabis sativa plant materials by fluid and/or gas extraction, for example by supercritical fluid extraction (SFE) with CO₂. The Cannabis sativa extract typically contains major cannabinoids, minor cannabinoids, terpenes, phytocannabinoids, and secondary metabolites. For example, the Cannabis sativa extract can include one or more of bisabolol, humulene, terpinene, caryophyllene, camphene, geraniol, guaiol, isopulegoll, ocimene, cymene, eucalyptol, terpinolene, and myrcene.

A “synthetic” cannabinoid or terpene is a cannabinoid or terpene made by chemical synthesis. The synthetic cannabinoid or terpene may be identical to a naturally occurring cannabinoid or terpene.

A “biosynthetic” cannabinoid or terpene is a cannabinoid or terpene made by a living organism or a laboratory process modeled after reactions in living organisms. The biosynthetic cannabinoid or terpene may be identical to a naturally occurring cannabinoid or terpene.

The term “MEM”, as used herein, refers to “minimal essential mixture” of the pharmaceutically active ingredient, pharmaceutical composition, or unit dosage of the present disclosure.

The term “E-MEM” as used herein, refers to “equimolar minimal essential mixture”

The term “DCR-MEM”, as used herein, refers to “Defined Cannabinoid-Ratio MEM”.

The practice of the present disclosure will employ, unless otherwise indicated, conventional methods of protein chemistry, biochemistry, recombinant DNA techniques and pharmacology, within the skill of the art. Such techniques are explained fully in the literature.

### 5.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 5.3 Pharmaceutically Active Ingredient

### 5.3.1. Cannabinoids

Accordingly, in a first aspect, active pharmaceutical ingredients (also referred to herein synonymously as “active ingredients” and “pharmaceutically active ingredients”) are provided that comprise a ratio of at least: cannabidiol (CBD), cannabidivarin (CBDV), and cannabichromene (CBC). In some embodiments, the active pharmaceutical ingredient comprises a ratio selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5.

In some embodiments, wherein the ratio is a molar ratio. In some embodiments, the active pharmaceutical ingredient comprises a molar ratio of: CBD, CBDV, and CBC. In some embodiments, the active pharmaceutical ingredient comprises a molar ratio selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 of CBD, CBDV, and CBC. In some embodiments, the active pharmaceutical ingredient comprises a molar ratio of 1:1:1 of CBD, CBDV, and CBC. In some embodiments, the active pharmaceutical ingredient comprises a molar ratio of 2:2:1 of CBD, CBDV, and CBC. In some embodiments, the active pharmaceutical ingredient comprises a molar ratio of 10:10:1 of CBD, CBDV, and CBC. In some embodiments, the active pharmaceutical ingredient comprises a molar ratio of 2:1:2 of CBD, CBDV, and CBC. In some embodiments, the active pharmaceutical ingredient comprises a molar ratio of 2:1:1 of CBD, CBDV, and CBC. In some embodiments, the active pharmaceutical ingredient comprises a molar ratio of 10:5:1 of CBD, CBDV, and CBC. In some embodiments, the active pharmaceutical ingredient comprises a molar ratio of 10:1:10 of CBD, CBDV, and CBC. In some embodiments, the active pharmaceutical ingredient comprises a molar ratio of 10:1:5 of CBD, CBDV, and CBC. In some embodiments, the active pharmaceutical ingredient comprises a molar ratio of 10:1:1 of CBD, CBDV, and CBC. In some embodiments, the active pharmaceutical ingredient comprises a molar ratio of 1:2:2 of CBD, CBDV, and CBC. In some embodiments, the active pharmaceutical ingredient comprises a molar ratio of 1:2:1 of CBD, CBDV, and CBC. In some embodiments, the active pharmaceutical ingredient comprises a molar ratio of 5:10:1 of CBD, CBDV, and CBC. In some embodiments, the active pharmaceutical ingredient comprises a molar ratio of 1:1:2 of CBD, CBDV, and CBC. In some embodiments, the active pharmaceutical ingredient comprises a molar ratio of 5:5:1 of CBD, CBDV, and CBC. In some embodiments, the active pharmaceutical ingredient comprises a molar ratio of 5:1:10 of CBD, CBDV, and CBC. In some embodiments, the active pharmaceutical ingredient comprises a molar ratio of 5:1:5 of CBD, CBDV, and CBC. In some embodiments, the active pharmaceutical ingredient comprises a molar ratio of 5:1:1 of CBD, CBDV, and CBC. In some embodiments, the active pharmaceutical ingredient comprises a molar ratio of 1:10:10 of CBD, CBDV, and CBC. In some embodiments, the active pharmaceutical ingredient comprises a molar ratio of 1:10:5 of CBD, CBDV, and CBC. In some embodiments, the active pharmaceutical ingredient comprises a molar ratio of 1:10:1 of CBD, CBDV, and CBC. In some embodiments, the active pharmaceutical ingredient comprises a molar ratio of 1:5:10 of CBD, CBDV, and CBC. In some embodiments, the active pharmaceutical ingredient comprises a molar ratio of 1:5:5 of CBD, CBDV, and CBC. In some embodiments, the active pharmaceutical ingredient comprises a molar ratio of 1:5:1 of CBD, CBDV, and CBC. In some embodiments, the active pharmaceutical ingredient comprises a molar ratio of 1:1:10 of CBD, CBDV, and CBC. In some embodiments, the active pharmaceutical ingredient comprises a molar ratio of 1:1:5 of CBD, CBDV, and CBC.

In some embodiments, the active pharmaceutical ingredient comprises a molar ratio selected from: 1-part CBD: 1-part CBDV: 1-part CBC; 2-parts CBD: 2-parts CBDV: 1-part CBC; 10-parts CBD: 10-parts CBDV: 1-part CBC; 2-parts CBD: 1 CBDV: 2-parts CBC; 2-parts CBD: 1-part CBDV:1-CBC; 10-parts CBD: 5-parts CBDV: 1-part CBC ; 10-parts CBD: 1-parts CBDV: 10-parts CBC; 10-parts CBD: 1-parts CBDV: 5-parts CBC; and 10-parts CBD: 1-part CBDV: 1-part CBC. In certain embodiments, the active pharmaceutical ingredient comprises a molar ratio selected from: 1-part CBD: 2-part CBDV: 2-part CBC; 1-part CBD: 2-parts CBDV: 1-part CBC; 5-parts CBD: 10-parts CBDV: 1-part CBC; 1-part CBD: 1-part CBDV: 2-parts CBC; 1-part CBD: 1-part CBDV:1 -part CBC; 5-parts CBD: 5-parts CBDV: 1-part CBC; 5-parts CBD: 1-part CBDV: 10-parts CBC; 5-parts CBD: 1-part CBDV: 5-parts CBC; and 5-parts CBD: 1-part CBDV: 1-part CBC.

In certain embodiments, the active pharmaceutical ingredient comprises a molar ratio selected from: 1-part CBD: 10-parts CBDV: 10-parts CBC; 1-part CBD: 10-parts CBDV: 5-parts CBC; 1-part CBD: 10-parts CBDV: 1-part CBC; 1-part CBD: 5-parts CBDV: 10-parts CBC; 1-part CBD: 5-parts CBDV:5-parts CBC; 1-part CBD: 5-part CBDV: 1-part CBC; 1-part CBD: 1-part CBDV: 10-parts CBC; 1-part CBD: 1-part CBDV: 5-parts CBC; and 1-part CBD: 1-part CBDV: 1-part CBC;

In certain embodiments, the molar ratio is 2:1:2 of CBD, CBDV, and CBC. In certain embodiments, the molar ratio is 1:1:1 of CBD, CBDV, and CBC.

In some embodiments, the cannabinoids in the pharmaceutically active ingredient are synthetic or biosynthetic compounds.

In another aspect, active pharmaceutical ingredients are provided that comprise a ratio of at least: cannabidiol (CBD), cannabichromene (CBC), and cannabidiol (CBN). In some embodiments, the ratio is selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of CBD, CBC and CBN.

In certain embodiments, the ratio is a molar ratio. In some embodiments, the ratio is a molar ratio of CBD, CBC, and CBN. In various embodiments, the molar ratio is selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; or 1:1:5 ratio of CBD, CBC and CBN. In some embodiments, the active pharmaceutical ingredient comprises a molar ratio of: 1:1:1 of CBD, CBC, and CBN. In some embodiments, the active pharmaceutical ingredient comprises a molar ratio of: 2:2:1 of CBD, CBC, and CBN. In some embodiments, the active pharmaceutical ingredient comprises a molar ratio of: 10: 10: 1 of CBD, CBC, and CBN. In some embodiments, the active pharmaceutical ingredient comprises a molar ratio of: 2:1:2 of CBD, CBC, and CBN. In some embodiments, the active pharmaceutical ingredient comprises a molar ratio of: 2:1:1 of CBD, CBC, and CBN. In some embodiments, the active pharmaceutical ingredient comprises a molar ratio of: 10:5:1 of CBD, CBC, and CBN. In some embodiments, the active pharmaceutical ingredient comprises a molar ratio of: 10:1:10 of CBD, CBC, and CBN. In some embodiments, the active pharmaceutical ingredient comprises a molar ratio of 10:1:5 of CBD, CBC, and CBN. In some embodiments, the active pharmaceutical ingredient comprises a molar ratio of 10:1:1 of CBD, CBC, and CBN. In some embodiments, the active pharmaceutical ingredient comprises a molar ratio of: 1:2:2 of CBD, CBC, and CBN. In some embodiments, the active pharmaceutical ingredient comprises a molar ratio of: 1:2:1 of CBD, CBC, and CBN. In some embodiments, the active pharmaceutical ingredient comprises a molar ratio of: 5:10:1 of CBD, CBC, and CBN. In some embodiments, the active pharmaceutical ingredient comprises a molar ratio of: 1:1:2 of CBD, CBC, and CBN. In some embodiments, the active pharmaceutical ingredient comprises a molar ratio of: 5:5:1 of CBD, CBC, and CBN. In some embodiments, the active pharmaceutical ingredient comprises a molar ratio of: 5:1:10 of CBD, CBC, and CBN. In some embodiments, the active pharmaceutical ingredient comprises a molar ratio of: 5:1:5 of CBD, CBC, and CBN. In some embodiments, the active pharmaceutical ingredient comprises a molar ratio of: 5:1:1 of CBD, CBC, and CBN. In some embodiments, the active pharmaceutical ingredient comprises a molar ratio of: 1:10:10 of CBD, CBC, and CBN. In some embodiments, the active pharmaceutical ingredient comprises a molar ratio of: 1:10:5 of CBD, CBC, and CBN. In some embodiments, the active pharmaceutical ingredient comprises a molar ratio of: 1:10:1 of CBD, CBC, and CBN. In some embodiments, the active pharmaceutical ingredient comprises a molar ratio of: 1:5:10 of CBD, CBC, and CBN. In some embodiments, the active pharmaceutical ingredient comprises a molar ratio of: 1:5:5 of CBD, CBC, and CBN. In some embodiments, the active pharmaceutical ingredient comprises a molar ratio of: 1:5:1 of CBD, CBC, and CBN. In some embodiments, the active pharmaceutical ingredient comprises a molar ratio of: 1:1:10 of CBD, CBC, and CBN. In some embodiments, the active pharmaceutical ingredient comprises a molar ratio of:1:1:5 of CBD, CBC, and CBN.

In some embodiments, the active pharmaceutical ingredient comprises a molar ratio selected from: 1-part CBD: 1-part CBN: 1-part CBC; 2-part CBD: 2-part CBN: 1-part CBC;10-part CBD: 10-part CBN: 1-part CBC; 2-part CBD: 1-part CBN: 2-part CBC; 2-part CBD: 1-part CBN: 1-part CBC; 10-part CBD: 5-part CBN: 1-part CBC; 10-part CBD: 1-part CBN: 10-part CBC; 10-part CBD: 1-part CBN: 5-part CBC; and 10-part CBD: 1-part CBN: 1-part CBC; 1-parts CBD: 2-part CBDV: 2-part CBC; 1-part CBD: 2-parts CBDV: 1-part CBC; 5-parts CBD: 10-parts CBDV: 1-part CBC; 1-part CBD: 1-part CBDV: 2-parts CBC; 1-part CBD: 1-part CBDV:1 -part CBC; 5-parts CBD: 5-parts CBDV: 1-part CBC; 5-parts CBD: 1-part CBDV: 10-parts CBC; 5-parts CBD: 1-part CBDV: 5-parts CBC; and 5-parts CBD: 1-part CBDV: 1-part CBC.

In some embodiments, the active pharmaceutical ingredient comprises a molar ratio selected from: 1-part CBD: 2-part CBN: 1-part CBC; 1-part CBD: 2-part CBN: 1-part CBC; 5-part CBD: 10-part CBN: 1-part CBC; 1-part CBD: 1-part CBN: 2-part CBC; 1-part CBD: 1-part CBN: 1-part CBC; 5-part CBD: 5-part CBN: 1-part CBC; 5-part CBD: 1-part CBN: 10-part CBC; 5-part CBD: 1-part CBN: 5-part CBC; and 5-part CBD: 1-part CBN: 1-part CBC.

In some embodiments, the active pharmaceutical ingredient comprises a molar ratio selected from: 1-part CBD: 10-part CBN: 10-part CBC; 1-part CBD: 10-part CBN: 5-part CBC; 1-part CBD: 10-part CBN: 1-part CBC; 1-part CBD: 5-part CBN: 10-part CBC; 1-part CBD: 5-part CBN: 5-part CBC; 1-part CBD: 5-part CBN: 1-part CBC; 1-part CBD: 1-part CBN: 10-part CBC; 1-part CBD: 1-part CBN: 5-part CBC; and 1-part CBD: 1-part CBN: 1-part CBC.

In some embodiments, the cannabinoids in the pharmaceutically active ingredient are synthetic or biosynthetic compounds.

In another aspect, active pharmaceutical ingredients are provided that comprise a ratio of at least: CBD, CBDV, and cannabigerol (CBG). In some embodiments, the active pharmaceutical ingredient comprises a ratio selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of: CBD, CBDV, and CBG. In some embodiments, the active pharmaceutical ingredient comprises a molar ratio of: 1:1:1 of CBD, CBDV, and CBG. In some embodiments, the active pharmaceutical ingredient comprises a molar ratio of: 2:2:1 of CBD, CBDV, and CBG. In some embodiments, the active pharmaceutical ingredient comprises a molar ratio of: 10:10:1 of CBD, CBDV, and CBG. In some embodiments, the active pharmaceutical ingredient comprises a molar ratio of: 2:1:2 of CBD, CBDV, and CBG. In some embodiments, the active pharmaceutical ingredient comprises a molar ratio of: 2:1:1 of CBD, CBDV, and CBG. In some embodiments, the active pharmaceutical ingredient comprises a molar ratio of: 10:5:1 of CBD, CBDV, and CBG. In some embodiments, the active pharmaceutical ingredient comprises a molar ratio of: 10:1:10 of CBD, CBDV, and CBG. In some embodiments, the active pharmaceutical ingredient comprises a molar ratio of 10:1:5 of CBD, CBDV, and CBG. In some embodiments, the active pharmaceutical ingredient comprises a molar ratio of: 10:1:1 of CBD, CBDV, and CBG. In some embodiments, the active pharmaceutical ingredient comprises a molar ratio of: 1:2:2 of CBD, CBDV, and CBG. In some embodiments, the active pharmaceutical ingredient comprises a molar ratio of: 1:2:1 of CBD, CBDV, and CBG. In some embodiments, the active pharmaceutical ingredient comprises a molar ratio of: 5:10:1 of CBD, CBDV, and CBG. In some embodiments, the active pharmaceutical ingredient comprises a molar ratio of: 1:1:2 of CBD, CBDV, and CBG. In some embodiments, the active pharmaceutical ingredient comprises a molar ratio of: 5:5:1 of CBD, CBDV, and CBG. In some embodiments, the active pharmaceutical ingredient comprises a molar ratio of: 5:1:10 of CBD, CBDV, and CBG. In some embodiments, the active pharmaceutical ingredient comprises a molar ratio of: 5:1:5 of CBD, CBDV, and CBG. In some embodiments, the active pharmaceutical ingredient comprises a molar ratio of: 5:1:1 of CBD, CBDV, and CBG. In some embodiments, the active pharmaceutical ingredient comprises a molar ratio of: 1:10:10 of CBD, CBDV, and CBG. In some embodiments, the active pharmaceutical ingredient comprises a molar ratio of: 1:10:5 of CBD, CBDV, and CBG. In some embodiments, the active pharmaceutical ingredient comprises a molar ratio of: 1:10:1 of CBD, CBDV, and CBG. In some embodiments, the active pharmaceutical ingredient comprises a molar ratio of: 1:5:10 of CBD, CBDV, and CBG. In some embodiments, the active pharmaceutical ingredient comprises a molar ratio of: 1:5:5 of CBD, CBDV, and CBG. In some embodiments, the active pharmaceutical ingredient comprises a molar ratio of: 1:5:1 of CBD, CBDV, and CBG. In some embodiments, the active pharmaceutical ingredient comprises a molar ratio of: 1:1:10 of CBD, CBDV, and CBG. In some embodiments, the active pharmaceutical ingredient comprises a molar ratio of: 1:1:5 of CBD, CBDV, and CBG.

In some embodiments, the ratio is a molar ratio. In certain embodiments, the ratio is a molar ratio of CBD, CBDV, and CBG. In some embodiments, the pharmaceutically active ingredient comprises a molar ratio selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of: CBD, CBDV, and CBG.

In certain embodiments, the ratio is a molar ratio of CBD, CBDV, and CBG. In some embodiments, the pharmaceutically active ingredient comprises a molar ratio selected from: 1-part CBD: 1-part CBDV: 1-part CBG; 2-part CBD: 2-part CBDV: 1-part CBG; 10-part CBD: 10-part CBDV: 1-part CBG; 2-part CBD: 1-part CBDV: 2-part CBG; 2-part CBD: 1-part CBDV: 1-part CBG; 10-part CBD: 5-part CBDV: 1-part CBG; 10-part CBD: 1-part CBDV: 10-part CBG; 10-part CBD: 1-part CBDV: 5-part CBG; and 10-part CBD: 1-part CBDV: 1-part CBG.

In some embodiments, the pharmaceutically active ingredient comprises a molar ratio selected from:1-part CBD:2 -part CBDV: 2-part CBG; 1-part CBD: 2-part CBDV: 1-part CBG; 5-part CBD: 10-part CBDV: 1-part CBG; 1-part CBD: 1-part CBDV: 2-part CBG; 1-part CBD: 1-part CBDV: 1-part CBG; 5-part CBD: 5-part CBDV: 1-part CBG; 5-part CBD: 1-part CBDV: 10-part CBG; 5-part CBD: 1-part CBDV: 5-part CBG; and 5-part CBD: 1-part CBDV: 1-part CBG.

In some embodiments, the pharmaceutically active ingredient comprises a molar ratio selected from: 1-part CBD: 10-part CBDV: 10-part CBG; 1-part CBD: 10-part CBDV: 5-part CBG; 1-part CBD: 10-part CBDV: 1-part CBG; 1-part CBD: 5-part CBDV: 10-part CBG; 1-part CBD: 5-part CBDV: 5-part CBG; 1-part CBD: 5-part CBDV: 1-part CBG; 1-part CBD: 1-part CBDV: 10-part CBG; 1-part CBD: 1-part CBDV: 5-part CBG; and 1-part CBD: 1-part CBDV: 1-part CBG.

In some embodiments, the cannabinoids in the pharmaceutically active ingredient are synthetic or biosynthetic compounds.

In various embodiments, the active ingredient is substantially free of tetrahydrocannabinol (THC). In some embodiments, the active ingredient is free of tetrahydrocannabinol (THC).

### 5.3.2. Terpenes

In some embodiments, the pharmaceutically active ingredient comprises one or more terpenes. In some embodiments, the one or more terpenes is selected from: alpha-pinene, trans-nerolidol, limonene, linalool and phytol. In certain embodiments, the one or more terpenes is alpha-pinene. In certain embodiments, the one or more terpenes is trans-nerolidol. In certain embodiments, the one or more terpenes is limonene. In certain embodiments, the one or more terpenes is linalool. In certain embodiments, the one or more terpenes is phytol. In certain embodiments, the one or more terpenes is beta-caryophyllene. In some embodiments, the one or more terpenes comprises limonene, linalool, nerolidol, pinene, and phytol.

In various embodiments, the active ingredient is substantially free of terpenes. In various embodiments, the active ingredient is free of terpenes.

In various embodiments, the active ingredient is substantially free of Alpha Pinene. In some embodiments, the active ingredient is free of Alpha-Pinene.

In various embodiments, the active ingredient is substantially free of Trans-Nerolidol. In various embodiments, the active ingredient is free of Trans-Nerolidol.

In various embodiments, the active ingredient is substantially free of Linalool. In some embodiments, the active ingredient is free of Linalool.

In various embodiments, the active ingredient is substantially free of limonene. In some embodiments, the active ingredient is free of limonene.

In various embodiments, the active ingredient is substantially free of phytol. In some embodiments, the active ingredient is free of phytol.

### 5.3.3. Relative Content

In typical embodiments, the cannabinoids collectively constitute 5-40% by weight (wt%) of the active ingredient.

In certain embodiments, the cannabinoids collectively constitute 5-10 wt% of the active ingredient, 10-15 wt% of the active ingredient, 15-20 wt% of the active ingredient, 20-25 wt% of the active ingredient, 25-30 wt% of the active ingredient, 30-35 wt% of the active ingredient, or 35-40 wt% of the active ingredient. In certain embodiments, the cannabinoids collectively constitute at least 5 wt%, at least 10 wt%, at least 15 wt%, at least 20 wt%, at least 25 wt%, at least 30 wt%, at least 35 wt%, but each case no more than 40 wt%, of the active ingredient.

In typical embodiments, the selected terpenes collectively constitute 0-70% by weight of the active ingredient. In embodiments in which at least one optional selected terpene is present, the selected terpenes collectively constitute 5-70 wt% of the active ingredient.

In certain embodiments, the selected terpenes collectively constitute at least 0 wt%, at least 1 wt%, at least 2 wt%, at least 3 wt%, at least 4 wt%, at least 5 wt%, at least 10 wt%, at least 15 wt%, at least 20 wt%, at least 25 wt%, at least 30 wt%, at least 35 wt%, at least 40 wt%, at least 45 wt%, at least 50 wt%, at least 55 wt%, at least 60 wt%, or at least 65 wt%, but in each case less than 70 wt%, of the active ingredient.

### 5.4.4. Absolute Content

In some embodiments, the pharmaceutically active ingredient comprises or consists of major cannabinoids (CBD or CBN), minor cannabinoids (cannabigerol (CBG), cannabichromene (CBC) and cannabidivarin (CBDV). In some embodiments, the pharmaceutically active ingredient comprises or consists of major cannabinoids (CBD or CBN), minor cannabinoids (cannabigerol (CBG), cannabichromene (CBC), cannabidivarin (CBDV), and optionally terpenes. In these embodiments, the major cannabinoids, minor cannabinoids, and optionally selected terpenes collectively constitute 100 wt% of the pharmaceutically active ingredient.

In some embodiments, the pharmaceutically active ingredient comprises or consists of major cannabinoids (CBD or CBN), minor cannabinoids (cannabigerol (CBG), cannabichromene (CBC) and cannabidivarin (CBDV), without terpenes. In these embodiments, the major cannabinoids, minor cannabinoids, without terpenes collectively constitute 100 wt% of the pharmaceutically active ingredient.

In some embodiments, the active ingredient consists essentially of major cannabinoids and minor cannabinoids.

In other embodiments, the major cannabinoids and minor cannabinoids collectively constitute less than 100% by weight (wt%) of the pharmaceutically active ingredient.

In some embodiments, the pharmaceutically active ingredient consists of major cannabinoids and minor cannabinoids. In these embodiments, the major cannabinoids and minor cannabinoids collectively constitute 100 % (w/v) of the pharmaceutically active ingredient.

In some embodiments, the active ingredient consists essentially of major cannabinoids and minor cannabinoids. In other embodiments, the major cannabinoids and minor cannabinoids collectively constitute less than 100% (w/v) of the pharmaceutically active ingredient.

### 5.4. Pharmaceutical Compositions

In another aspect, pharmaceutical compositions are provided. The pharmaceutical composition comprises the pharmaceutically active ingredient disclosed herein and a pharmaceutically acceptable carrier or diluent.

In one aspect, the pharmaceutical composition comprises a pharmaceutically active ingredient comprising a ratio (e.g., molar ratio) of: CBD, CBDV, and CBC and a pharmaceutically acceptable carrier or diluent.

In another aspect, the pharmaceutical composition comprises a pharmaceutically active ingredient comprising a ratio (e.g., molar ratio) of: CBD, CBC, and CBN, and a pharmaceutically acceptable carrier or diluent.

In another aspect, the pharmaceutical composition comprises a pharmaceutically active ingredient comprising a ratio (e.g., molar ratio) of: CBD, CBDV, and CBG, and a pharmaceutically acceptable carrier or diluent.

### 5.4.1. Content of Pharmaceutically Active Ingredient

In typical embodiments, the active ingredient is present in the pharmaceutical composition at a concentration of at least 0.0001 mg/ml, at least 0.001 mg/ml, at least 0.01 mg/ml, at least 0.1 mg/ml, at least 0.5 mg/ml, or at least 1 mg/ml. In certain embodiments, the active ingredient is present in the pharmaceutical composition at a concentration of at least 1 mg/ml, at least 2 mg/ml, at least 3 mg/ml, at least 4 mg/ml, at least 5 mg/ml, at least 10 mg/ml, at least 15 mg/ml, at least 20 mg/ml, or at least 25 mg/ml. In certain embodiments, the active ingredient is present in the pharmaceutical composition at a concentration of at least 30 mg/ml, at least 35 mg/ml, at least 40 mg/ml, at least 45 mg/ml or at least 50 mg/ml. In certain embodiments, the active ingredient is present in the pharmaceutical composition at a concentration of at least 55 mg/ml, at least 60 mg/ml, at least 65 mg/ml, at least 70 mg/ml, at least 80 mg/ml, at least 90 mg/ml, or at least 100 mg/ml.

### 5.4.2. Formulation Generally

The pharmaceutical composition can be in any form appropriate for human or veterinary medicine, including a liquid, an oil, an emulsion, a gel, a colloid, an aerosol or a solid.

The pharmaceutical composition can be formulated for administration by any route of administration appropriate for human or veterinary medicine, including enteral and parenteral routes of administration.

In various embodiments, the pharmaceutical composition is formulated for oral administration. In certain embodiments, the pharmaceutical composition is formulated or oral administration as an oral disintegrating tablet.

In various embodiments, the pharmaceutical composition is formulated for sublingual administration. In various embodiments, the pharmaceutical composition is formulated for buccal administration.

In some embodiments, the pharmaceutical composition is administered p.r.n.

In various embodiments, the pharmaceutical composition is formulated for administration by inhalation. In certain of these embodiments, the pharmaceutical composition is formulated for administration by a vaporizer. In certain of these embodiments, the pharmaceutical composition is formulated for administration by a nebulizer. In certain of these embodiments, the pharmaceutical composition is formulated for administration by an aerosolizer.

In various embodiments, the pharmaceutical composition is formulated for administration by a nanoparticle or nanoemulsion encapsulating the active ingredient.

In certain embodiments, the pharmaceutical composition can be encapsulated via nanoparticles, which may improve their stability. In certain embodiments, nanoparticles may be in a polymer matrix. In certain embodiments, the nanoparticles are lipid nanoparticles, that have a lipid monolayer enclosing a solid lipid core, dendrimers (nano sized three-dimensional branched molecules of polymer), nanotubes (sequence of nanoscale C60 atoms arranged in a long thin cylindrical structure), or nanoshells (concentric sphere consisting of a dielectric core and a metal shell).

In some embodiments, nanoemulsions include sub-micron sized emulsions. Nanoemulsion is an emulsion system having the droplet size in nanometer scale in which oil or water droplets are finely dispersed in the opposite phase with the help of a suitable surfactant to stabilize the system. The average droplet size usually ranges from 0.1 to 500 nm. The size of the droplets varies depending on the drug particles, mechanical energy, composition and relative amount of the surfactants. Nanoemulsions are also known as miniemulsions, fine-dispersed emulsions, submicron emulsions etc., which can be either O/W (oil in water) or W/O (water in oil) emulsion.

In various embodiments, the pharmaceutical composition is formulated for oral administration. In some embodiments, the pharmaceutical composition is formulated for intravenous, intramuscular, or subcutaneous administration. In some embodiments, the pharmaceutical composition is formulated for intrathecal or intracerebroventricular administration. In some embodiments, the pharmaceutical composition is formulated for topical administration.

### 5.4.3. Pharmacological Compositions Adapted for Oral/Buccal/Sublingual Administration

Formulations for oral, buccal or sublingual administration may be in the form of capsules, cachets, pills, tablets (e.g., such as, but not limited to, oral disintegrating tablets), lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject polypeptide therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral, buccal or sublingual administration (capsules, tablets, pills, dragees, powders, granules, and the like), one or more therapeutic agents may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents. In some embodiments, the oral composition is an oral disintegrating tablet.

In some embodiments, unit dosage forms of the active pharmaceutical ingredient described herein are provided that are adapted for administration of the pharmaceutical composition by vaporizer, nebulizer, or aerosolizer. In some embodiments, the dosage form is a vial, an ampule, ly scored to allow user opening. In particular embodiments, the nebulizer is a jet nebulizer or an ultrasonic nebulizer.

Inhalable compositions are generally administered in an aqueous solution e.g., as a nasal or pulmonary spray. Preferred systems for dispensing liquids as a nasal spray are disclosed in U.S. Pat. No. 4,511,069. Such formulations may be conveniently prepared by dissolving compositions according to the present disclosure in water to produce an aqueous solution, and rendering the solution sterile. The formulations may be presented in multi-dose containers, for example in the sealed dispensing system disclosed in U.S. Pat. Nos. 4,511,069. Other suitable nasal spray delivery systems have been described in Transdermal Systemic Medication, Y. W. Chien Ed., Elsevier Publishers, New York, 1985; M. Naef et al. Development and pharmacokinetic characterization of pulmonal and intravenous delta-9-tetrahydrocannabinol (THC) in humans, J. Pharm. Sci. 93, 1176-84 (2004); and in U.S. Pat. Nos. 4,778,810; 6,080,762; 7,052,678; and 8,277,781 (each incorporated herein by reference). Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Mucosal formulations are administered as dry powder formulations e.g., comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 micron mass median equivalent aerodynamic diameter (MMEAD), commonly about 1 micron MMEAD, and more typically about 2 micron MMEAD. Maximum particle size appropriate for deposition within the nasal passages is often about 10 micron MMEAD, commonly about 8 micron MMEAD, and more typically about 4 micron MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient’s breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

### 5.4.4. Pharmacological Compositions Adapted for Injection

For intravenous, intramuscular, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer’s Injection, Lactated Ringer’s Injection. Preservatives, stabilizers, buffers, antioxidants and/or other additives can be included, as required.

In various embodiments, the unit dosage form is a vial, ampule, bottle, or pre-filled syringe. In some embodiments, the unit dosage form contains 0.01 mg, 0.1 mg, 0.5 mg, 1 mg, 2.5 mg, 5 mg, 10 mg, 12.5 mg, 25 mg, 50 mg, 75 mg, or 100 mg of the cannabinoid composition. In some embodiments, the unit dosage form contains 125 mg, 150 mg, 175 mg, or 200 mg of the cannabinoid composition. In some embodiments, the unit dosage form contains 250 mg of the cannabinoid composition.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 1 ml, 2.5 ml, 5 ml, 7.5 ml, 10 ml, 25 ml, or 50 ml of pharmaceutical composition.

In particular embodiments, the unit dosage form is a vial containing 1 ml of the cannabinoid composition at a concentration of 0.01 mg/ml, 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or 26 mg/ml, 27 mg/ml, 28 mg/ml, 29 mg/ml, 30 mg/ml or 30 mg/ml. In some embodiments, the unit dosage form is a vial containing 2 ml of the cannabinoid composition at a concentration of 0.01 mg/ml, 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or 26 mg/ml, 27 mg/ml, 28 mg/ml, 29 mg/ml, 30 mg/ml or 40 mg/ml.

In some embodiments, the pharmaceutical composition in the unit dosage form is in solid form, such as a lyophilate, suitable for solubilization.

Unit dosage form embodiments suitable for subcutaneous, intradermal, or intramuscular administration include preloaded syringes, auto-injectors, and autoinject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an autoinject pen. The autoinject pen comprises an autoinject pen containing a pharmaceutical composition as described herein. In some embodiments, the autoinject pen delivers a predetermined volume of pharmaceutical composition. In other embodiments, the autoinject pen is configured to deliver a volume of pharmaceutical composition set by the user.

In various embodiments, the autoinject pen contains about 0.1 mL to about 5.0 mL of the pharmaceutical composition. In specific embodiments, the autoinject pen contains about 0.5 mL of the pharmaceutical composition. In particular embodiments, the autoinject pen contains about 1.0 mL of the pharmaceutical composition. In other embodiments, the autoinject pen contains about 5.0 mL of the pharmaceutical composition.

### 5.4.5. Pharmacological Compositions Adapted for Topical Administration

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the cannabinoid-containing complex mixtures featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). The cannabinoid-containing complex mixtures featured in the disclosure may be encapsulated within liposomes or may form complexes thereto, in particular to cationic liposomes. Alternatively, the cannabinoid-containing complex mixtures may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-10 alkyl ester (e.g., isopropylmyristate IPM), monoglyceride, diglyceride or pharmaceutically acceptable salt thereof.

### 5.5. Dose Ranges, Generally

In vivo and/or in vitro assays may optionally be employed to help identify optimal dosage ranges for use. The precise dose to be employed in the formulation will also depend on the route of administration, and the seriousness of the condition, and should be decided according to the judgment of the practitioner and each subject’s circumstances. Effective doses may be extrapolated from dose-response curves derived from in vitro or animal model test systems.

### 5.6. Unit Dosage Forms

The active pharmaceutical ingredient may conveniently be presented in unit dosage form.

In some embodiments, each of the cannabinoids of the unit dosage form is present at a concentration of at least 0.01 µM when administered. In some embodiments, each of the cannabinoids of the unit dosage form is present at a concentration of at least 0.1 µM when administered. In some embodiments, each of the cannabinoids of the unit dosage form is present at a concentration of at least 0.25 µM when administered. In some embodiments, each of the cannabinoids of the unit dosage form is present at a concentration of at least 0.5 µM when administered. In some embodiments, each of the cannabinoids of the unit dosage form is present at a concentration of at least 0.1 µM, at least 0.2 µM, at least 0.3 µM, at least 0.4 µM, at least 0.5 µM, at least 0.5 µM, at least 0.6 µM, at least 0.7 µM, at least 0.8 µM, at least 0.9 µM, at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at leaset 12 µM, at least 13 µM, at least 14 µM, at least 15 µM, at least 16 µM, at least 17 µM, at least 18 µM, at least 19 µM, at least 20 µM, at least 22 µM, at least 24 µM, at least 26 µM, at least 28 µM, at least 30 µM, at least 32 µM, at least 34 µM, at least 36 µM, at least 38 µM, at least 40 µM, at least 42 µM, at least 44 µM, at least 46 µM, at least 48 µM, or at least 50 µM, when administered. In some embodiments, each of the cannabinoids of the unit dosage form is present at a concentration ranging from at least 0.1-50 µM (e.g., such as 0.25-3 µM, 0.1-10 µM, 0.1 µM to 20 µM, 0.1 µM to 30 µM, 0.1 µM to 40 µM, 0.25 µM to 40 µM, 0.25 µM to 45 µM, etc.) when administered. In some embodiments, each of the cannabinoids of the unit dosage form is present at a concentration ranging from at least 0.25-3 µM when administered. In some embodiments, each of the cannabinoids of the unit dosage form is present at a concentration ranging from at least 0.25-4 µM when administered. In some embodiments, each of the cannabinoids of the unit dosage form is present at a concentration ranging from at least 0.1-3 µM when administered. In some embodiments, each of the cannabinoids of the unit dosage form is present at a concentration ranging from at least 0.1-4 µM when administered.

In some embodiments, one of the cannabinoids of the unit dosage form is CBD and is present at a concentration ranging from at least 0.1-50 µM (e.g., such as 0.25-3 µM, 0.1-10 µM, 0.1 µM to 20 µM, 0.1 µM to 30 µM, 0.1 µM to 40 µM, 0.25 µM to 40 µM, 0.25 µM to 45 µM, etc.) when administered. In some embodiments, one of the cannabinoids of the unit dosage form is CBD and is present at a concentration ranging from at least 0.1-4 µM. In some embodiments, one of the cannabinoids of the unit dosage form is CBD and is present at a concentration ranging from at least 0.1-3 µM. In some embodiments, one of the cannabinoids of the unit dosage form is CBD and is present at a concentration ranging from at least 0.25-4 µM. In some embodiments, one of the cannabinoids of the unit dosage form is CBD and is present at a concentration ranging from at least 0.25-3 µM. In some embodiments, one of the cannabinoids of the unit dosage form is CBD and is present at a concentration of at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at least 15 µM, at least 20 µM, at least 25 µM, at least 30 µM, at least 35 µM, at least 40 µM, at least 45 µM, or at least 50 µM, when administered.

In some embodiments, one of the cannabinoids of the unit dosage form is CBN and is present at a concentration ranging from at least 0.1-50 µM (e.g., such as 0.25-3 µM, 0.1-10 µM, 0.1 µM to 20 µM, 0.1 µM to 30 µM, 0.1 µM to 40 µM, 0.25 µM to 40 µM, 0.25 µM to 45 µM, etc.) when administered. In some embodiments, one of the cannabinoids of the unit dosage form is CBN and is present at a concentration ranging from at least 0.1-4 µM. In some embodiments, one of the cannabinoids of the unit dosage form is CBN and is present at a concentration ranging from at least 0.1-3 µM. In some embodiments, one of the cannabinoids of the unit dosage form is CBN and is present at a concentration ranging from at least 0.25-4 µM. In some embodiments, one of the cannabinoids of the unit dosage form is CBN and is present at a concentration ranging from at least 0.25-3 µM. In some embodiments, one of the cannabinoids of the unit dosage form is CBN and is present at a concentration of at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at least 15 µM, at least 20 µM, at least 25 µM, at least 30 µM, at least 35 µM, at least 40 µM, at least 45 µM, or at least 50 µM, when administered.

In some embodiments, one of the cannabinoids of the unit dosage form is CBC and is present at a concentration ranging from at least 0.1-50 µM (e.g., such as 0.25-3 µM, 0.1-10 µM, 0.1 µM to 20 µM, 0.1 µM to 30 µM, 0.1 µM to 40 µM, 0.25 µM to 40 µM, 0.25 µM to 45 µM, etc.) when administered. In some embodiments, one of the cannabinoids of the unit dosage form is CBC and is present at a concentration ranging from at least 0.1-4 µM. In some embodiments, one of the cannabinoids of the unit dosage form is CBC and is present at a concentration ranging from at least 0.1-3 µM. In some embodiments, one of the cannabinoids of the unit dosage form is CBC and is present at a concentration ranging from at least 0.25-4 µM. In some embodiments, one of the cannabinoids of the unit dosage form is CBC and is present at a concentration ranging from at least 0.25-3 µM. In some embodiments, one of the cannabinoids of the unit dosage form is CBC and is present at a concentration of at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at least 15 µM, at least 20 µM, at least 25 µM, at least 30 µM, at least 35 µM, at least 40 µM, at least 45 µM, or at least 50 µM, when administered.

In some embodiments, one of the cannabinoids of the unit dosage form is CBG and is present at a concentration ranging from at least 0.1-50 µM (e.g., such as 0.25-3 µM, 0.1-10 µM, 0.1 µM to 20 µM, 0.1 µM to 30 µM, 0.1 µM to 40 µM, 0.25 µM to 40 µM, 0.25 µM to 45 µM, etc.) when administered. In some embodiments, one of the cannabinoids of the unit dosage form is CBG and is present at a concentration ranging from at least 0.1-4 µM. In some embodiments, one of the cannabinoids of the unit dosage form is CBG and is present at a concentration ranging from at least 0.1-3 µM. In some embodiments, one of the cannabinoids of the unit dosage form is CBG and is present at a concentration ranging from at least 0.25-4 µM. In some embodiments, one of the cannabinoids of the unit dosage form is CBG and is present at a concentration ranging from at least 0.25-3 µM. In some embodiments, one of the cannabinoids of the unit dosage form is CBG and is present at a concentration of at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at least 15 µM, at least 20 µM, at least 25 µM, at least 30 µM, at least 35 µM, at least 40 µM, at least 45 µM, or at least 50 µM, when administered.

In some embodiments, one of the cannabinoids of the unit dosage form is CBDV and is present at a concentration ranging from at least 0.1-50 µM (e.g., such as 0.25-3 µM, 0.1-10 µM, 0.1 µM to 20 µM, 0.1 µM to 30 µM, 0.1 µM to 40 µM, 0.25 µM to 40 µM, 0.25 µM to 45 µM, etc.) when administered. In some embodiments, one of the cannabinoids of the unit dosage form is CBDV and is present at a concentration ranging from at least 0.1-4 µM. In some embodiments, one of the cannabinoids of the unit dosage form is CBDV and is present at a concentration ranging from at least 0.1-3 µM. In some embodiments, one of the cannabinoids of the unit dosage form is CBDV and is present at a concentration ranging from at least 0.25-4 µM. In some embodiments, one of the cannabinoids of the unit dosage form is CBDV and is present at a concentration ranging from at least 0.25-3 µM. In some embodiments, one of the cannabinoids of the unit dosage form is CBDV and is present at a concentration of at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at least 15 µM, at least 20 µM, at least 25 µM, at least 30 µM, at least 35 µM, at least 40 µM, at least 45 µM, or at least 50 µM, when administered.

In some embodiments, each of the cannabinoids of the unit dosage form is present in an amount sufficient to achieve a mean peak concentration (Cmax) in plasma and/or target tissue of at least 0.1 µM when administered. In some embodiments, each of the cannabinoids of the unit dosage form is present in an amount sufficient to achieve a mean peak concentration (Cmax) in plasma and/or target tissue of at least 0.25 µM when administered. In some embodiments, each of the cannabinoids of the unit dosage form is present in an amount sufficient to achieve a mean peak concentration (Cmax) in plasma and/or target tissue of: at least 0.1 µM, at least 0.25 µM, at least 5 µM, at least 1 µM, at least 2 µM, at least 3 µM, at least 4 µM, at least 5 µM, at least 10 µM, at least 15 µM, at least 20 µM, at least 25 µM, at least 30 µM, at least 35 µM, at least 40 µM, at least 45 µM, or at least 50 µM, when administered. In certain embodiments, CBD of the unit dosage form is present in an amount sufficient to achieve a mean peak concentration (Cmax) in plasma and/or target tissue of: at least 0.1 µM, at least 0.25 µM, at least 5 µM, at least 1 µM, at least 2 µM, at least 3 µM, at least 4 µM, at least 5 µM, at least 10 µM, at least 15 µM, at least 20 µM, at least 25 µM, at least 30 µM, at least 35 µM, at least 40 µM, at least 45 µM, or at least 50 µM, when administered. In certain embodiments, CBN of the unit dosage form is present in an amount sufficient to achieve a mean peak concentration (Cmax) in plasma and/or target tissue of: at least 0.1 µM, at least 0.25 µM, at least 5 µM, at least 1 µM, at least 2 µM, at least 3 µM, at least 4 µM, at least 5 µM, at least 10 µM, at least 15 µM, at least 20 µM, at least 25 µM, at least 30 µM, at least 35 µM, at least 40 µM, at least 45 µM, or at least 50 µM, when administered. In certain embodiments, CBC of the unit dosage form is present in an amount sufficient to achieve a mean peak concentration (Cmax) in plasma and/or target tissue of: at least 0.1 µM, at least 0.25 µM, at least 5 µM, at least 1 µM, at least 2 µM, at least 3 µM, at least 4 µM, at least 5 µM, at least 10 µM, at least 15 µM, at least 20 µM, at least 25 µM, at least 30 µM, at least 35 µM, at least 40 µM, at least 45 µM, or at least 50 µM, when administered. In certain embodiments, CBG of the unit dosage form is present in an amount sufficient to achieve a mean peak concentration (Cmax) in plasma and/or target tissue of: at least 0.1 µM, at least 0.25 µM, at least 5 µM, at least 1 µM, at least 2 µM, at least 3 µM, at least 4 µM, at least 5 µM, at least 10 µM, at least 15 µM, at least 20 µM, at least 25 µM, at least 30 µM, at least 35 µM, at least 40 µM, at least 45 µM, or at least 50 µM when administered. In certain embodiments, CBDV of the unit dosage form is present in an amount sufficient to achieve a mean peak concentration (Cmax) in plasma and/or target tissue of: at least 0.1 µM, at least 0.25 µM, at least 5 µM, at least 1 µM, at least 2 µM, at least 3 µM, at least 4 µM, at least 5 µM, at least 10 µM, at least 15 µM, at least 20 µM, at least 25 µM, at least 30 µM, at least 35 µM, at least 40 µM, at least 45 µM, or at least 50 µM, when administered.

A target tissue can be any tissue or organ in the body, such as, but not limited to, heart, lungs, liver, kidney, and brain. In some embodiments, the target tissue is the tissue or organ that is affected by Parkinson’s Disease, including Parkinson’s Movement Disorder.

. In some embodiments, the total unit dosage form of the pharmaceutically active ingredient or pharmaceutical composition comprising two or more, or three or more cannabinoids is at a concentration of at least 0.1 µM, at least 0.2 µM, at least 0.3 µM, at least 0.4 µM, at least 0.5 µM, at least 0.5 µM, at least 0.6 µM, at least 0.7 µM, at least 0.8 µM, at least 0.9 µM, at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at leaset 12 µM, at least 13 µM, at least 14 µM, at least 15 µM, at least 16 µM, at least 17 µM, at least 18 µM, at least 19 µM, at least 20 µM, at least 22 µM, at least 24 µM, at least 26 µM, at least 28 µM, at least 30 µM, at least 32 µM, at least 34 µM, at least 36 µM, at least 38 µM, at least 40 µM, at least 42 µM, at least 44 µM, at least 46 µM, at least 48 µM, at least 50 µM, at least 55 µM, at least 60 µM, at least 70 µM, at least 80 µM, at least 90 µM, at least 100 µM, at least 110 µM, at least 120 µM, at least 130 µM, at least 140 µM, at least 150 µM, at least 160 µM, at least 170 µM, or at least 180 µM, when administered.

. In some embodiments, the total unit dosage form of the pharmaceutically active ingredient or pharmaceutical composition comprising CBD, CBDV, and CBC is at a concentration of at least 0.1 µM, at least 0.2 µM, at least 0.3 µM, at least 0.4 µM, at least 0.5 µM, at least 0.5 µM, at least 0.6 µM, at least 0.7 µM, at least 0.8 µM, at least 0.9 µM, at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at leaset 12 µM, at least 13 µM, at least 14 µM, at least 15 µM, at least 16 µM, at least 17 µM, at least 18 µM, at least 19 µM, at least 20 µM, at least 22 µM, at least 24 µM, at least 26 µM, at least 28 µM, at least 30 µM, at least 32 µM, at least 34 µM, at least 36 µM, at least 38 µM, at least 40 µM, at least 42 µM, at least 44 µM, at least 46 µM, at least 48 µM, at least 50 µM, at least 55 µM, at least 60 µM, at least 70 µM, at least 80 µM, at least 90 µM, at least 100 µM, at least 110 µM, at least 120 µM, at least 130 µM, at least 140 µM, at least 150 µM, at least 160 µM, at least 170 µM, or at least 180 µM, when administered.

In some embodiments, the total unit dosage form of the pharmaceutically active ingredient or pharmaceutical composition comprising CBD, CBN, and CBC is at a concentration of at least 0.1 µM, at least 0.2 µM, at least 0.3 µM, at least 0.4 µM, at least 0.5 µM, at least 0.5 µM, at least 0.6 µM, at least 0.7 µM, at least 0.8 µM, at least 0.9 µM, at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at leaset 12 µM, at least 13 µM, at least 14 µM, at least 15 µM, at least 16 µM, at least 17 µM, at least 18 µM, at least 19 µM, at least 20 µM, at least 22 µM, at least 24 µM, at least 26 µM, at least 28 µM, at least 30 µM, at least 32 µM, at least 34 µM, at least 36 µM, at least 38 µM, at least 40 µM, at least 42 µM, at least 44 µM, at least 46 µM, at least 48 µM, at least 50 µM, at least 55 µM, at least 60 µM, at least 70 µM, at least 80 µM, at least 90 µM, at least 100 µM, at least 110 µM, at least 120 µM, at least 130 µM, at least 140 µM, at least 150 µM, at least 160 µM, at least 170 µM, or at least 180 µM, when administered.

In some embodiments, the total unit dosage form of the pharmaceutically active ingredient or pharmaceutical composition comprising CBD, CBDV, and CBG is at a concentration of at least 0.1 µM, at least 0.2 µM, at least 0.3 µM, at least 0.4 µM, at least 0.5 µM, at least 0.5 µM, at least 0.6 µM, at least 0.7 µM, at least 0.8 µM, at least 0.9 µM, at least 1 µM, at least 1.5 µM, at least 2 µM, at least 2.5 µM, at least 3 µM, at least 3.5 µM, at least 4 µM, at least 4.5 µM, at least 5 µM, at least 5.5 µM, at least 6 µM, at least 6.6 µM, at least 7 µM, at least 7.5 µM, at least 8 µM, at least 8.5 µM, at least 9 µM, at least 9.5 µM, at least 10 µM, at leaset 12 µM, at least 13 µM, at least 14 µM, at least 15 µM, at least 16 µM, at least 17 µM, at least 18 µM, at least 19 µM, at least 20 µM, at least 22 µM, at least 24 µM, at least 26 µM, at least 28 µM, at least 30 µM, at least 32 µM, at least 34 µM, at least 36 µM, at least 38 µM, at least 40 µM, at least 42 µM, at least 44 µM, at least 46 µM, at least 48 µM, at least 50 µM, at least 55 µM, at least 60 µM, at least 70 µM, at least 80 µM, at least 90 µM, at least 100 µM, at least 110 µM, at least 120 µM, at least 130 µM, at least 140 µM, at least 150 µM, at least 160 µM, at least 170 µM, or at least 180 µM, when administered.

In certain embodiments, the unit dosage form comprises at least 0.001 mg, at least 0.01 mg, at least 0.02 mg, at least 0.03 mg, at least 0.04 mg, at least 0.05 mg, at least 0.06 mg, at least 0.07 mg, at least 0.08 mg, at least 0.09 mg, at least 1 mg, at least 2 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, at least 20 mg, at least 21 mg, at least 22 mg, at least 23 mg, at least 24 mg, at least 25 mg, at least 26 mg, at least 27 mg, at least 28 mg, at least 29 mg, at least 30 mg, at least 31 mg, at least 32 mg, at least 33 mg, at least 34 mg, at least 35 mg, at least 40 mg, at least 45 mg, at least 50 mg, at least 55 mg, or at least 60 mg dosage of each of the cannabinoids in the pharmaceutical composition.

In some embodiments, the unit dosage form comprises 0.2 mg to 40 mg of CBD, 0.1 mg to 20 mg of CBDV, and 0.2 mg to 40 mg of CBC. In some embodiments, the unit dosage form comprises 0.1 mg to 20 mg of CBD, 0.1 mg to 20 mg of CBN, and 0.1 mg to 20 mg of CBC. In some embodiments, the unit dosage form comprises 0.1 mg to 20 mg of CBD, 0.2 mg to 40 mg of CBDV, and 0.1 mg to 20 mg of CBG. In some embodiments, the unit dosage form comprises 0.01 mg to 5 mg of CBD, 0.02 mg to 10 mg of CBDV, and 1 mg to 50 mg of CBG.

In certain embodiments, the total unit dosage form of the pharmaceutically active ingredient or pharmaceutical composition thereof comprises at least 1 mg, at least 2 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, at least 20 mg, at least 21 mg, at least 22 mg, at least 23 mg, at least 24 mg, at least 25 mg, at least 26 mg, at least 27 mg, at least 28 mg, at least 29 mg, at least 30 mg, at least 31 mg, at least 32 mg, at least 33 mg, at least 34 mg, at least 35 mg, at least 40 mg, at least 45 mg, at least 50 mg, at least 55 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, or at least 180 mg dosage.

### 5.7. Methods of Treatment

In another aspect, methods are presented for treating a subject having a disease responsive to the cannabinoid-containing minimal essential mixtures (MEMs) described herein. The method comprises administering to the subject a therapeutically effective amount of cannabinoid-containing minimal essential mixtures (MEMs) described herein.

In certain embodiment, the disease responsive to an active ingredient or pharmaceutical composition described herein is a disease of the brain or central nervous system (CNS). In some embodiments, the disease is a neurodegenerative disease. In some embodiments, the disease is Alzheimer’s disease, Parkinson’s disease, including Parkinsonian Movement Disorder and dopamine-related Parkinson’s Disease symptoms, Lewy Body Dementia, or Huntington’s disease. In certain embodiments, the disease responsive to an active ingredient or pharmaceutical composition described herein is Parkinson’s disease, and the active ingredient or pharmaceutical composition is administered to a patient in amount effective to reduce or treat a dopamine-related Parkinson’s Disease symptom.

In a particular aspect, the present disclosure provides methods of treating a patient who has, or who is at risk for developing, Parkinson’s Disease, including Parkinsonian Movement Disorder. In some embodiment, the patient has Parkinsonian Movement Disorder. In some embodiments, the patient has dopamine-related Parkinson’s disease symptoms. In some embodiments, a patient who has Parkinsonian Movement Disorder or dopamine-related Parkinson’s Disease symptoms experiences tremors, Bradykinesia (slowness of movement), dizziness or fainting, drooling, dyskinesia, dystonia, facial asking, postural instability (trouble with balance and falls), rigidity, stooped posture, and/or trouble moving or walking. Therefore, the methods of the present disclosure provide methods of treating symptoms associated with Parkinson Movement disorder such as, but not limited to: tremors, Bradykinesia (slowness of movement), dizziness or fainting, drooling, dyskinesia, dystonia, facial asking, postural instability (trouble with balance and falls), rigidity, stooped posture, and/or trouble moving or walking.

Another aspect of the present disclosure relates to a method of treating a dopamine-related Parkinson’s Disease symptom by administering an effective amount of the pharmaceutical composition disclosed herein to a patient having a dopamine-related Parkinson’s Disease symptom. In such embodiments, the patient has Parkinson’s Disease. In some embodiments, the dopamine-related Parkinson’s Disease symptom comprises one or more of: tremor, rigidity, slowness of movement, and impaired balance and coordination. In some embodiments, the dopamine-related Parkinson’s Disease symptom comprises one or more of: dopamine-related difficulties in concentrating, poor coordination, stooped posture, loss of smell, disruptions in the reward centers of the brain, anxiety and depression.

In certain embodiments, the dopamine-related Parkinson’s Disease symptom comprises anxiety. In certain embodiments, the dopamine-related Parkinson’s Disease symptom comprises depression. In certain embodiments, the dopamine-related Parkinson’s Disease symptom comprises tremors. In certain embodiments, the dopamine-related Parkinson’s Disease symptom comprises rigidity. In certain embodiments, the dopamine-related Parkinson’s Disease symptom comprises slowness of movement. In certain embodiments, the dopamine-related Parkinson’s Disease symptom comprises loss of balance and coordination. In certain embodiments, the dopamine-related Parkinson’s Disease symptom comprises loss of smell. In certain embodiments, the dopamine-related Parkinson’s Disease symptom comprises one or more of: dopamine-related difficulties in concentrating, poor coordination, stooped posture, and disruptions in the reward centers of the brain.

In typical embodiments, the cannabinoid and/or terpene-containing minimal essential mixtures (MEMs) are administered in the form of a pharmaceutical composition as described above. These methods are particularly aimed at therapeutic and prophylactic treatments of animals, and more particularly, humans.

The terms “treatment”, “treating”, and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

By the term “therapeutically effective dose” or “effective amount” is meant a dose or amount that produces the desired effect for which it is administered. The exact dose or amount will depend on the purpose of the treatment, and will be ascertainable by one skilled in the art using known techniques (see, e.g., Lloyd (1999) The Art, Science and Technology of Pharmaceutical Compounding).

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

The term “ameliorating” refers to any therapeutically beneficial result in the treatment of a disease state.

The actual amount administered, and rate and time-course of administration, will depend on the nature and severity of disease being treated. Prescription of treatment, e.g. decisions on dosage etc., is within the responsibility of general practitioners and other medical professionals, and typically takes account of the disorder to be treated, the condition of the individual patient, the site of delivery, the method of administration and other factors known to practitioners. Examples of the techniques and protocols mentioned above can be found in Remington’s Pharmaceutical Sciences, 16th edition, Osol, A. (ed), 1980.

In some embodiments, the effective amount of the active pharmaceutical ingredient, pharmaceutical composition, or unit dose is administered orally, by inhalation, by buccal administration, by sublingual administration, by injection or by topical application.

In some embodiments, the effective amount of the active pharmaceutical ingredient, pharmaceutical composition, or unit dose in an amount sufficient to treat a person who has, or is at risk for developing, Parkinson’s Disease, including Parkinsonian Movement Disorder.

In some embodiments, the cannabinoids in the pharmaceutically active ingredient, pharmaceutical composition or unit dose are administered in an amount less than 1 g, less than 500 mg, less than 100 mg, less than 10 mg per dose. In some embodiments, the cannabinoids in the pharmaceutically active ingredient, pharmaceutical composition or unit dose are administered in an amount of 1 g per dose, 500 mg per dose, 100 mg per dose, or 10 mg per dose.

In some embodiments, the active pharmaceutical ingredient, pharmaceutical composition, or unit dose is administered once a day, 2-4 times a day, 2-4 times a week, once a week, or once every two weeks.

A composition can be administered alone or in combination with other treatments, either simultaneously or sequentially dependent upon the condition to be treated.

### 6. EXAMPLES

### 6.1. Example 1: Minimum Essential Therapeutic Mixtures of Cannabinoids in Cell and Animal Models of Parkinson’s Disease

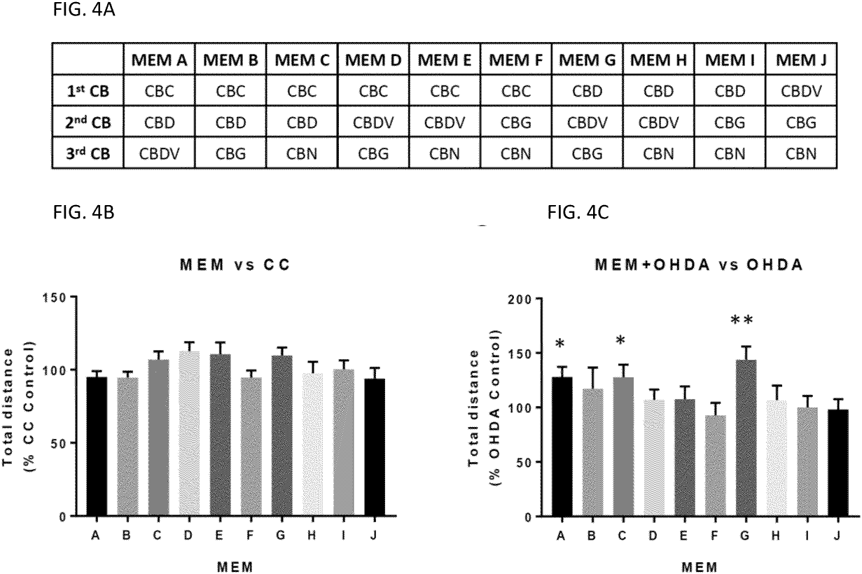

This study aimed to further reduce the number of compounds in formulations to a minimal essential mixture (MEM) that could recapitulate as many of the effects of the original combination of MEM A, MEM B, MEM C, MEM D, MEM E, MEM F, and MEM I, MEM J (see e.g. FIG. 4A) as possible with the goal of generating a mixture that would be more amenable to pharmacological production.

Two cell assays were initially used to evaluate the potential therapeutic efficacy of the mixtures (MEM A through MEM J) by using both an in vitro neuroprotection assay and a dopamine secretion assay in dopaminergic neuronal cell models. From these cell assays, the cannabinoids as being largely responsible for the activity seen was identified in a mixture (CBG, CBC, CBDV, alpha-pinene, trans-nerolidol, limonene, linalool and phytol) with a nominal effect of the terpenes.

Then, multiple drug combinations that contained three individual cannabinoids were assayed for their ability to ameliorate a 6-hydroxydopmamine (OHDA)-induced model of PD in zebrafish larvae. The results allowed to move sequentially from the remarkable chemical complexity of the Cannabis plant, to moderately minimal essential mixtures (MEMs) with potential PD-therapeutic activity as evaluated in cell models, to refined minimal essential mixtures of cannabinoids that demonstrate therapeutic effects on OHDA treated zebrafish. The sequentially reductionist process used in this study preserved some of the entourage-like effects of whole plant extracts, while achieving ‘relative’ simplicity within MEM that is a requirement for obtaining the manufacturing and quality control advantages of single ingredient drugs.

### Materials and Methods

### Chemicals and Cell Lines

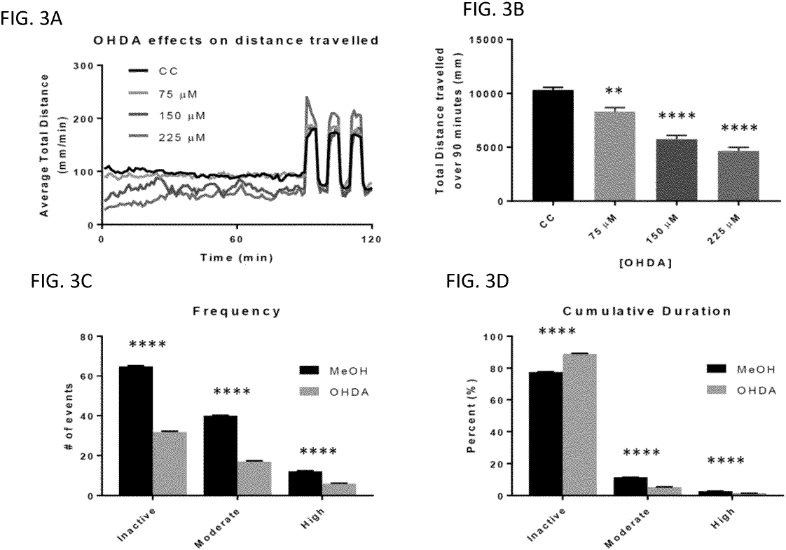

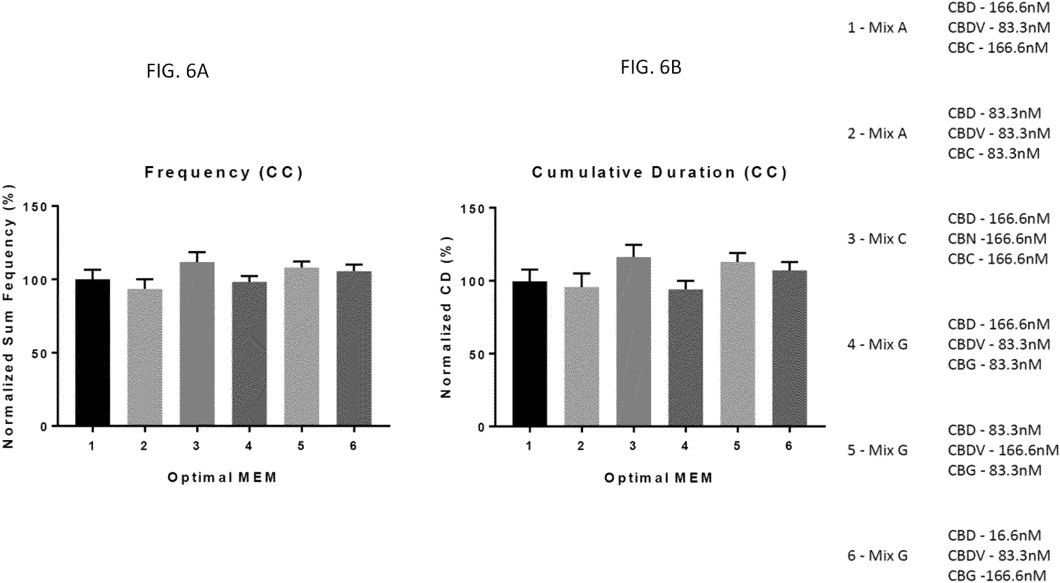

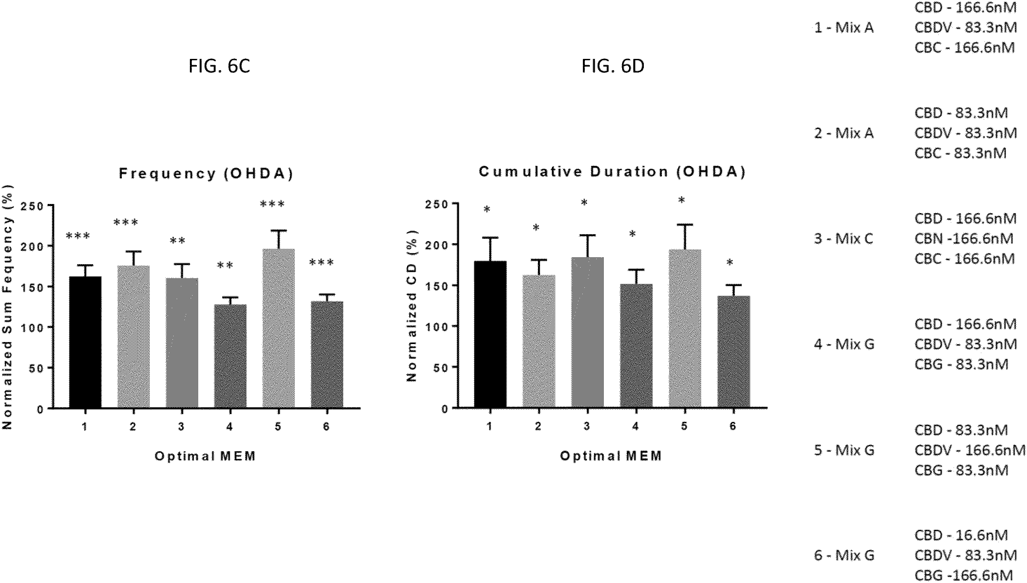

All cannabinoids used in this study were purchased as 1 mg/ml standards in methanol (Sigma, Ontario, Canada): Cannabidiol (CBD), Cannabichromene (CBC), Cannabidivarin (CBDV), Cannabigerol (CBG) and Cannabinol (CBN). The α-pinene (98% purity), trans-nerolidol (>85% purity), and Methanol (99.9% purity) were also purchased from Sigma (Sigma, Ontario, Canada). D-Limonene (96.9% purity) was purchased from MPBIO (MP Biomedicals LLC, Ohio, USA), Linalool (>96% purity) was purchased from TCI (TCI, Oregon, USA) and Phytol was from Agilent Technologies (Agilent Technologies, Inc., Rhode Island, USA). All terpenes were diluted in methanol. 6-hydroxydopamine (OHDA) was purchased from Sigma (Sigma, Ontario, Canada) and was diluted in saline buffer (0.9% NaCl) supplemented with 0.02% Ascorbic acid. Mixtures produced for cell line experiments used equimolar components as follows: MIX-1 = minor cannabinoids (CBC, CBG and CBDV) + Terpenes (Linalool, a-pinene, limonene, t-nerolidol and phytol), MIX-2 = terpenes only, and MIX-3 = Minor cannabinoids only. Individual major cannabinoids, CBD and CBN, were added at the same equimolar amount. FIG. 3 and FIG. 6 provide specifics regarding mixture compositions.

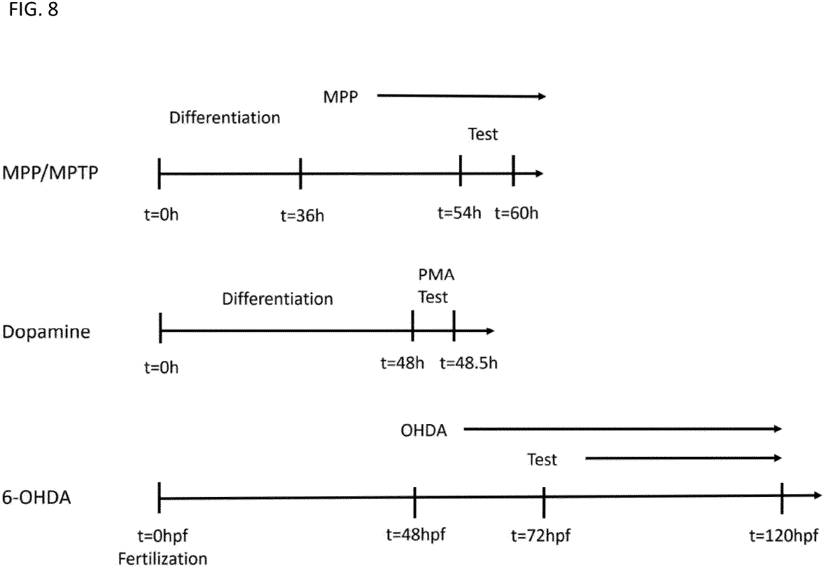

Cell lines used were from ATCC. Cath.a cells (sp. = mouse, cat# CRL-11179, ATCC, Manassas, Virginia, US), a CNS catecholaminergic cell line, were cultured according to ATCC instructions and were induced to CAD differentiated status by serum deprivation (0.5% FBS culture for 36 h) prior to experiments as described (Qi et al., 1997, Neurosci, 17, 1217-25). PC12 (sp. = rat, ATCC #CRL-1721) were cultured in RPMI 10% FBS. PC12 differentiation used Minimal Essential M medium containing 1% HS and 0.5% FBS, then the cells were treated with 100 ng/ml NGF, 100 ng/ml basic fibroblast growth factor (bFGF), and serum-starved media containing 2 mg/ml BSA for 2 days (Joen et al., 2010. Synapse, 64, pgs 765-72). Schematic representations of exposure paradigms are provided in FIG. 8.

### In Vitro Neuroprotection Assays

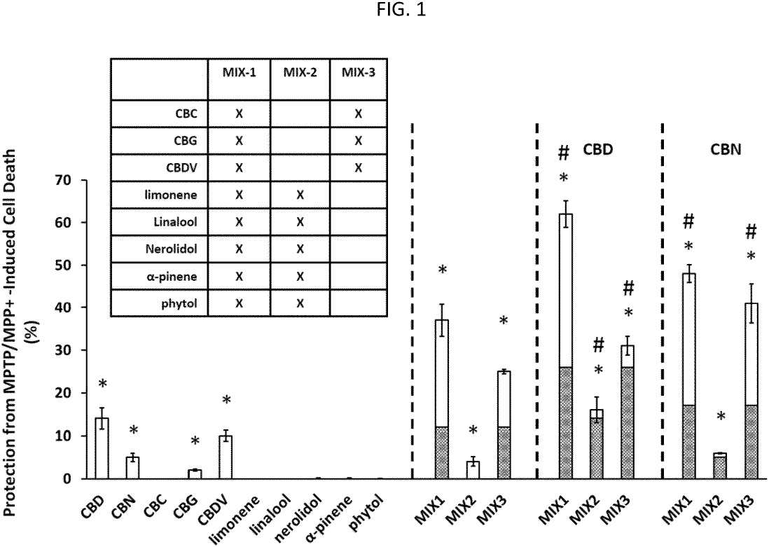

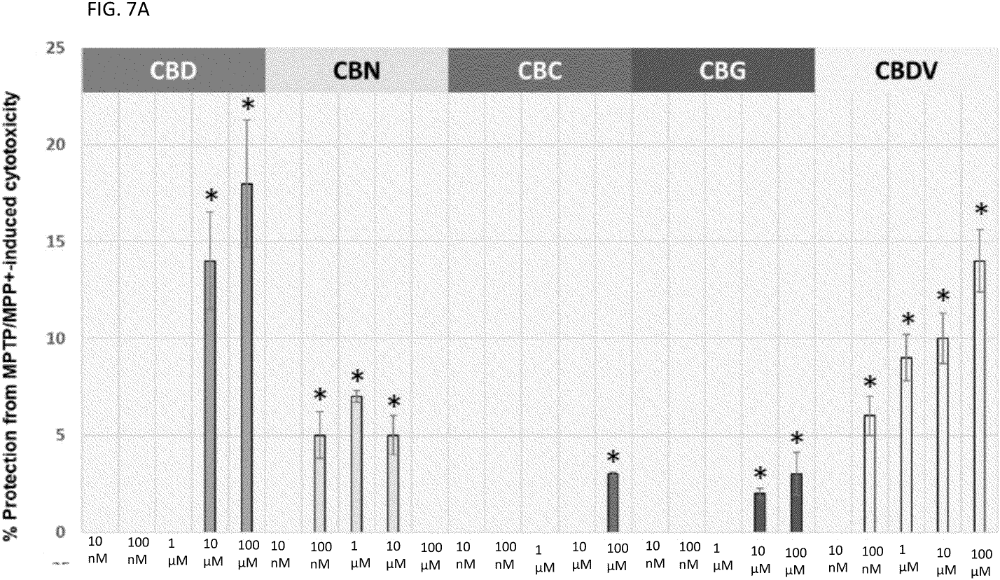

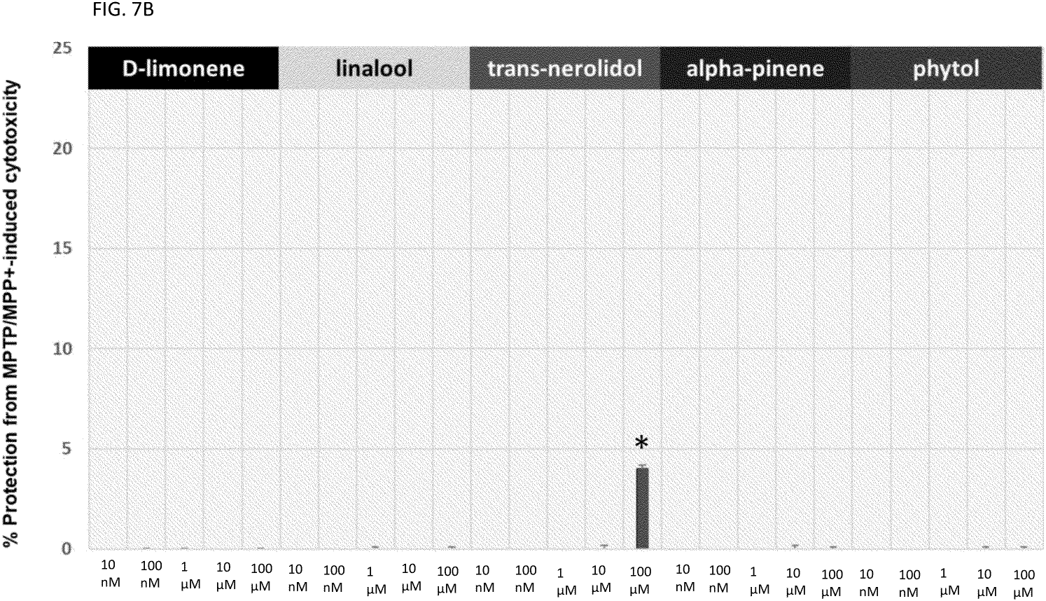

Neuroprotective effects were assessed based on the ability of both individual compounds and mixtures of compounds to protect against neuronal cell death induced by 1-methyl-4-phenylpyridinium (MPP+) in the 1-methyl-4-phenyl-1,2,3,6-tetrahydropyridine (MPTP)-based selective cytotoxicity assay (Arshad et al., 2014, Acta Biochim Biophys Sin (Shanghai), 46, 22-30). MPP+ is an active metabolite of MPTP that is known to cause human Parkinsonism after injection. MPTP/MPP+ assays were performed in vitro on Cath.a cells by applying each compound or mixture of compounds to the cell cultures 18 hours after application of MPP (Arshad et al., 2014, Acta Biochim Biophys Sin (Shanghai), 46, 22-30). Cell viability assessments were performed using a standard MTT cell viability assay (MTT Cell Proliferation Assay Kit, Cayman Chemicals, Ann Arbor, Michigan, Item No. 10009365). Cell viability was assessed 24 h after exposure to MPP, which is 6 h after exposure to the tested compound or compound mixture. Percent protection was normalized to MPTP control alone (100 % cell death). To establish an effective dose range across the compounds in this assay system, the neuroprotective effects of each individual compound were tested at 5 different concentrations (FIGS. 7A-7B). Based on these results the individual and equimolar mixtures were tested in the cell assays at 10 µM each (FIG. 1). Equimolar mixtures contained 10 µM of each compound. In all cases vehicle controls contained methanol at equal concentrations to those found in test compounds/mixtures, ≤ 5%.

### In Vitro Dopamine-Release Assay

In parallel with the neuroprotection assays, cannabinoid and terpene compounds were tested alone and in mixtures to determine their effects on dopamine release from differentiated PC12 cells. PC12 cells were differentiated as in (Joen et al., 2010. Synapse, 64, pgs 765-72; Hu et al., 2018, Int J Mol Med, 41, 195-201). Supernatant samples were collected from 3 replicate wells 30 mins after application of PMA/Ionomycin (positive control) or the indicated compounds, and dopamine was measured in the medium using the Dopamine ELISA Kit #KA1887 from Abnova (Abnova, California, US) according to manufacturer’s instructions. For dopamine secretion, dopamine release was normalized to PMA/ionomycin control (0% baseline).

### Zebrafish

A larval zebrafish model was selected to assess the ability of the cannabinoid mixtures to alleviate the movement disorders associated with Parkinson’s disease. When larval zebrafish are exposed to 6-hydroxydopamine (OHDA), there is a dose-dependent effect that leads to the inactivation and eventual death of DA-producing cells of the substantia nigra. DA-producing neuronal cell loss and associated DA depletion in the striatum are correlated with altered motor behavior and changes in the movement patterns of zebrafish. Zebrafish larval locomotion is often divided into burst swimming (high velocity) and slow swimming (lower velocity) states. Not surprisingly, these disparate behaviors have been mapped to multiple different regions of the brain that are enervated substantially by dopaminergic neurons.

The fish used in this study were wild-type AB/Tubingen hybrids. Age-matched embryos were reared in Pentair Aquatic Ecosystem (Apopka, Florida, USA) nursery baskets (200 embryos per basket) on a ZebTec Recirculation Water Treatment System (Tecniplast, Buguggiate, Varese, Italy) at 28.5 ± 0.5° C., on a 14-h day-10-h night light cycle. All adult zebrafish husbandry and breeding was in accordance with the Canadian Council of Animal Care guidelines.

### Behavioral Testing in Zebrafish

All compounds were diluted in 100% methanol (MeOH) and experiments were performed in a HEPES buffered E3 (HE3) medium (5 mM NaCl, 0.17 mM KCl, 0.33 mM CaCl2-2H2O, 0.33 mM MgSO4-7H2O, 10 mM HEPES, pH 7.2). Individual 120 hours post fertilization (hpf) larval zebrafish were transferred to a 48-well microtiter plate in 500 µL of HE3 media. Larvae were acclimated for at least one hour in a lighted 28.5° C. incubator (photon flux: 3-5 µmol s-1 m2) prior to experimentation and larval behavior was analyzed using DanioVision larval tracking systems with EthoVisionXT14/15 software (Noldus Information Technology Inc., Virginia, USA). Distance traveled was measured using dynamic subtraction at 28.5° C. over 120 minutes with the first 90 minutes under lighted conditions (15 µmol m-2 s-1), followed by alternating 5-minute dark/light cycles. Each larvae represents an independent measurement. Any larvae that were dead or displayed phenotypic abnormalities were removed from analysis. 12 larvae were used in each experimental condition, and at least 2 replicates of each concentration were performed.

### 6-Hydroxydopamine Parkinson’s Model Development and Advanced Behavioral Analysis

Larvae were exposed to varying concentrations of 6-hydroxydopamine (OHDA) from 48-120 hours post fertilization (hpf). 15 dechorionated larvae were transferred in 5 mL of HE3 to each well of a six-well plate. The 5 mL exposure media was replaced daily, and ascorbic acid/saline buffer (used to resuspend OHDA) was used as a vehicle control. Larvae were then loaded into 48-well microtiter plate in 500 µL of HE3 media as described above. Schematic representations of exposure paradigms shown in FIG. 8.

In addition to distance travelled, activity was measured as a percentage change in pixel density during data acquisition. The integrated visualization feature in EthoVision software (Noldus Information Technology Inc., Virginia, USA) was used to detect larval activity during three distinct activity types: high (greater than 0.5% pixel change per sampling), moderate (between 0.03% and 0.5%), and inactive (less than 0.03%) states. The frequency with which larvae switched between activity states and the cumulative duration of time spent in each activity state was then measured. Metrics were captured in 1 min bins, and the average over 90 minutes was used to calculate each metric in each activity state. For frequency calculations, the number of times larvae switched between activity states (high, moderate, and low) was calculated (termed Total Frequency). The cumulative amount of time spent in the high and moderate activity states combined (termed Cumulative Duration) was averaged over 90 minutes and measured as a percentage.

### Equimolar Minimum Essential Mixture (E-MEM) and Defined Cannabinoid-Ratio MEM (DCR-MEM)

### Testing Using OHDA PD Model

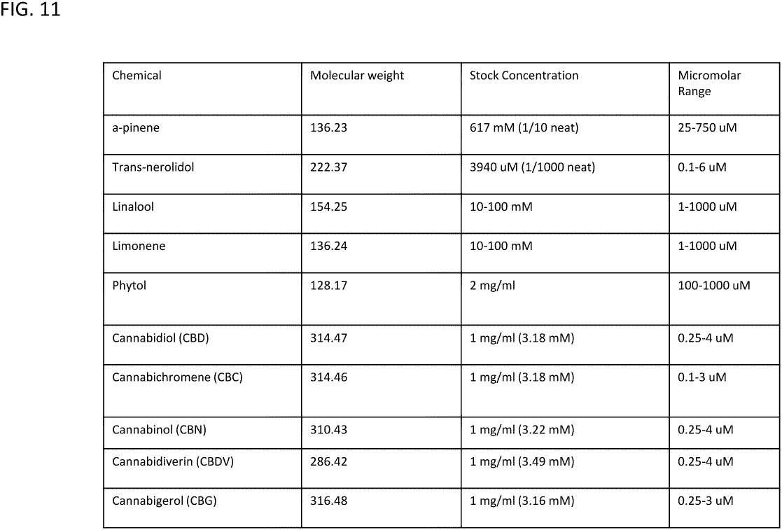

Acute behavioral assays were performed on the cannabinoids selected for the study as described above except larvae were loaded into the well plates with 450 µl HE3. Immediately prior to recording 50 µl of 10 × test compound solution was added to each well. For the OHDA challenge experiments 150 µM OHDA was used for all challenge experiments. During media replacements at 72 and 96 hpf, 25 µl of 200X stock solutions of the respective cannabinoid or E-MEM was added to the exposure media. All compounds were diluted in 100% methanol (MeOH) unless otherwise indicated. All experiments were run at least in duplicate unless stated. The chemical, stock concentration, and dosage range is provided as Table 4 of FIG. 11.

At 120 hpf larvae were washed with HE3 and transferred in 500 µl to 48-well plates. Behavioral analysis was then performed as described above. All experiments were performed at least in duplicate. The E-MEM selected for further study (above) were combined in non-equimolar ratios (defined cannabinoid-ratios DCR-MEM) and subjected to both the total distance and activity analysis metrics, as described above.

This procedure was done in 3 steps, reducing a single component by 50% (a 1:2 ratio relative to their original equimolar concentrations) or 90% (a 1:10 ratio relative to their original equimolar concentrations), reducing 2 components by 50% (1:2) or 90% (1:10), or a defined cannabinoid-ratio (DCR)-MEM reducing 2 components by different cannabinoid-ratios (1:2 or 1:10 relative to their original equimolar concentrations).

### Analysis and Statistical Methods

The mean +/- the standard deviations were calculated for all samples in the cell assays. A two-tailed student’s t-test was used to evaluate the statistical differences between sample types. Calculation of statistical significance for total distance traveled over 90 minutes was performed by one-way ANOVA using a Dunnett’s multiple comparison test using GraphPad Prism 7.04 software (La Jolla, CA). Comparisons of either OHDA + Drug vs OHDA, or Drug vs MeOH were performed as an unpaired Student’s t-test. An asterisk is used to represent a p-value less than 0.05 or lower, unless otherwise defined.

### Results

### Neuroprotective Effects of Cannabis-Compounds Alone and in Combination

At 10 µM, all of the individual cannabinoids except CBC displayed some neuroprotective effects while none of the terpenes were able to prevent MPTP/MPP+ induced apoptosis (FIG. 1). A mixture of the minor cannabinoids and the terpenes (MIX-1) showed a substantial neuroprotective effect with an increase in cell survival of 37 +/- 3.8%. Mixtures of either the terpenes alone (MIX-2) or the minor cannabinoids (MIX-3) were then created to assess their contribution to the overall activity of MIX-1. MIX-2 (terpenes) showed a limited overall protection (4 +/- 1.1%) while MIX-3 (minor cannabinoids) demonstrated similar activity to MIX-1 (25 +/- 0.5%). The effects of the major cannabinoids were then assessed by adding each individually to the mixtures. CBD increased cell survival in all three mixtures (62 +/- 3.1% vs 37 +/- 3.8% (MIX1), 16 +/- 3% vs 4 +/- 1.1% (MIX-2) and 31 +/- 2.2% vs 25 +/- 0.5% (MIX-3).

The effects of the second major cannabinoid (CBN) were similar but less pronounced than those seen for CBD. In all cases the mixtures were more neuroprotective than would have been estimated from the sum (hashed shading area of bars of FIG. 1) of their individual effects.

### Effects of Cannabis-Compounds in Mixtures on Dopamine-Release Responses

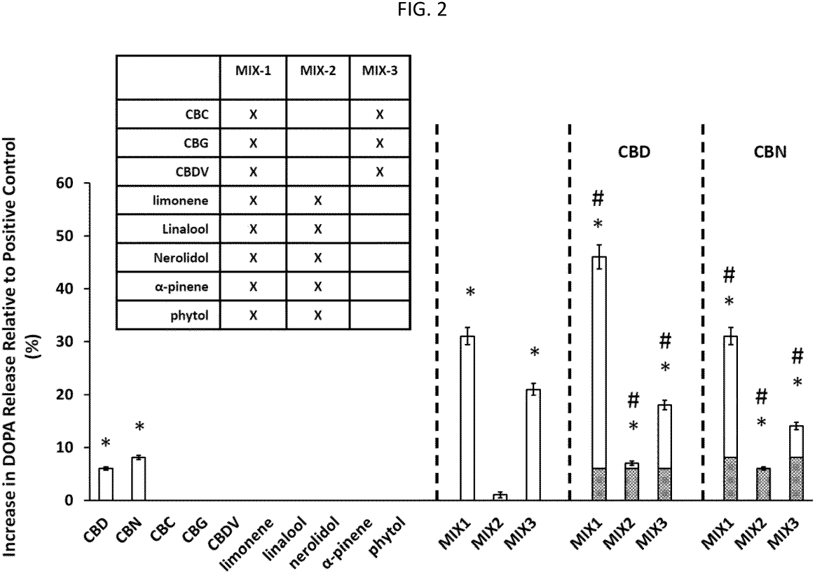

In parallel with the MPTP testing described previously, the effects of the same individual compounds and mixtures were tested on dopamine release from PC 12 cells (FIG. 2). The major cannabinoids, CBD (6.0 +/- 0.3% DOPA release) and CBN (8.1 +/- 0.4% DOPA release), were the only individual compounds tested that produced a statistically significant (p < 0.05) increase in dopamine release. The relative performance of the mixtures in the dopamine assay was similar to the trend observed in the MPTP/MPP assay. MIX-1 led to the largest increase in DOPA release of 31 +/- 1.6%, followed by MIX-3 (21 +/- 1.1%), while the DOPA release for MIX-2 was not significant. Again, the effectiveness of adding the major cannabinoids (CBD or CBN) was also evaluated to each MIX relative to the effectiveness of each MIX alone. When CBD was added to MIX-1, the effectiveness was increased leading to a DOPA release of 46 +/- 2.3% for MIX-1 + CBD, while adding CBN to MIX-1 did not significantly increase the DOPA release. The addition of CBD or CBN to MIX-2 were able to produce modest but significant increases in dopamine secretion up to 7.0 +/- 0.4% DOPA release for MIX-2 + CBD and 6.0 +/- 0.3% DOPA release for MIX-2 + CBN. Similarly, the addition of CBD to MIX-3 produced an increase in DOPA release relative to MIX-3 alone (MIX-3 + CBD produced 18 +/-0.9% DOPA release), while the addition of CBN to MIX-3 produced a significant reduction in the DOPA release compared to MIX-3 alone (MIX-3 + CBD produced 14 +/- 0.7% DOPA release). As in the neuroprotection assays, mixtures of the components were able to elicit significantly more robust responses than the individual components.

Taken together, these results demonstrate that the effects of the mixtures cannot be attributed to a single ingredient. On the contrary, it suggests that interactions between the components in the mixtures are critical for the maximal efficacy of the mixture. In addition, it appears from the cell assay data that the terpene components of the mixtures have a minimal contribution on their efficacy and that the cannabinoid components are sufficient to use as a potential therapeutic.

### Assessment of Cannabinoid Effects in a Zebrafish OHDA Parkinson’s Model

In order to test whether the cannabinoids and cannabinoid-based mixtures identified by the cell screening assays would potentially alleviate symptomatic effects in an animal model of Parkinson’s disease, a previously developed zebrafish larval model of dopamine cell loss that caused by exposure to 6-hydroxydopamine (OHDA) was applied (Feng et al., 2014, Zebrafish, 11, 227-39; Cronin and Grealy, 2017, Neuroscience, 367, 34-46; Benvenutti et al., 2018, PeerJ, 6, e4957.) and refined it based on a determination of OHDA dose and time conditions. It was found that larval exposure to OHDA produced a concentration-dependent decrease in the baseline activity during an initial 90-minute period in the light (FIGS. 3A and 3B). It also led to a non-significant increase in the maximum response to the light/dark transition (startle response) at 225 µM. Based on these findings, 150 µM OHDA was selected as the testing model as it produced a decrease in activity during the 90- minute baseline period, which can be considered a model of bradykinesia, while not impacting the startle response which suggests a minimal effect on general locomotor function (FIG. 3). Visual assessment of the OHDA treated larvae appeared to show a more complex pattern of behavior than could be assessed by a simple measure of distance travelled. It was observed that OHDA treated larvae, when at rest, displayed a small, periodic side-to-side movement with no velocity that may represent a ‘resting tremor’. This behavior had not been previously defined and further highlights the significance of the OHDA exposure to act as a model of PD. Thus, in addition to analyzing distance travelled by the larvae as described above, larval activity was also analyzed using a % pixel-change based assessment (FIGS. 3C and 3D). The activity was divided into three types: high, moderate, and inactive states. The high and moderate activities reflect burst swimming (escape behavior) and slower speed foraging swimming respectively (Budick and O′Malley, 2000, J Exp Biol, 203, 2565-79.). The inactive state was set between 0 and 0.03% change as a way to quantify the ‘resting tremor’ as a unique phenotype in the OHDA treated zebrafish larvae. OHDA treatment caused a reduction in the frequency of all states (FIG. 3C). Typically, OHDA treatment resulted in a 50-60% reduction in activity state transitions for all three states (FIG. 3C). As shown in FIG. 3D, all larvae spent the majority of their time in the inactive state, with untreated larvae spending ~80% of their activity in the inactive state, ~15% in the moderately active state and ~5% in the highly active state (FIG. 3D). The OHDA treated larvae spent a greater fraction of their time in the inactive state (~90%) with a concomitant decrease in moderate and high activities (5 and 1% respectively).

### Assessment of Behavioral Response to Cannabinoids and Equimolar MEMs

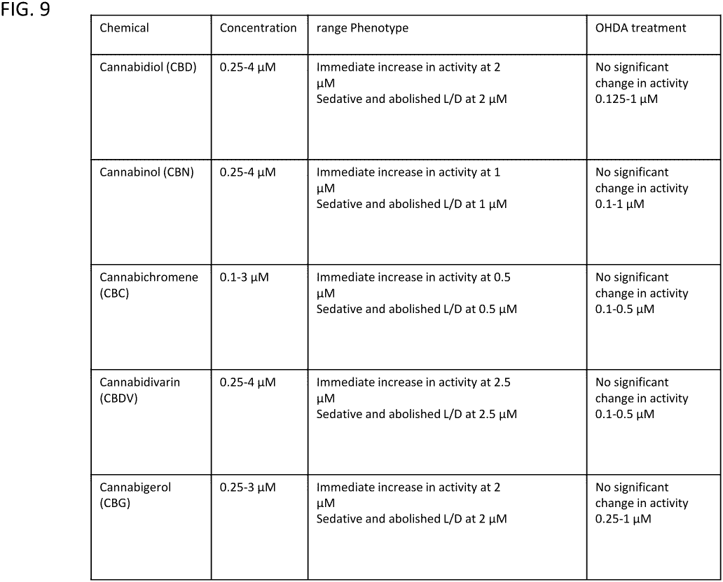

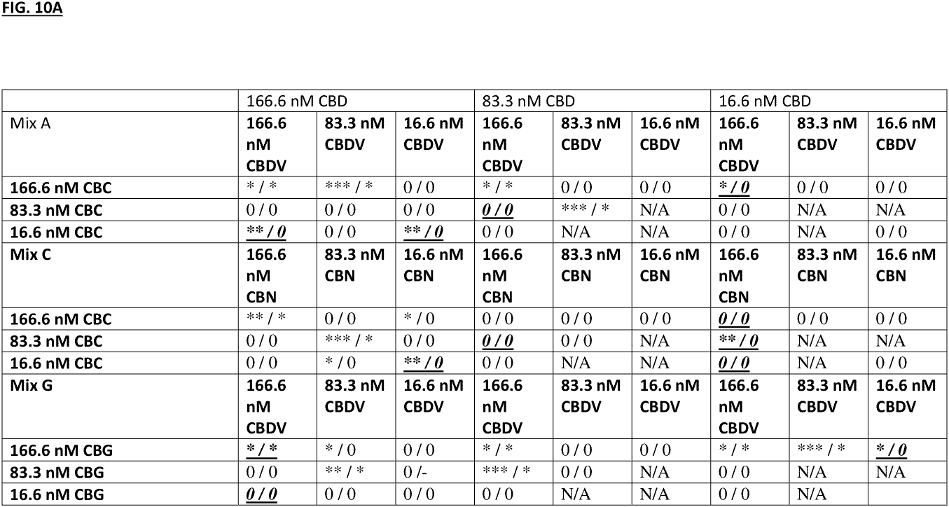

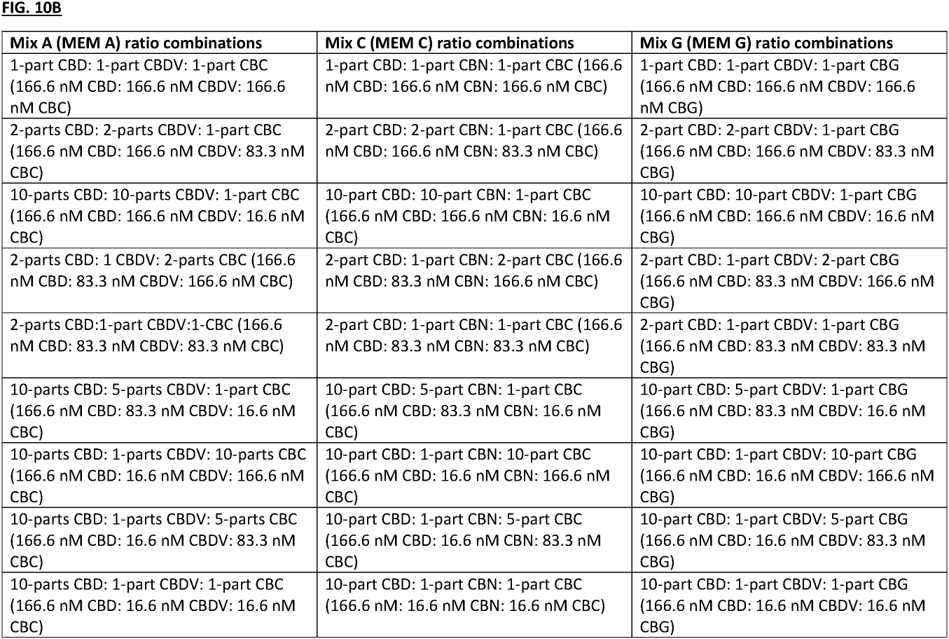

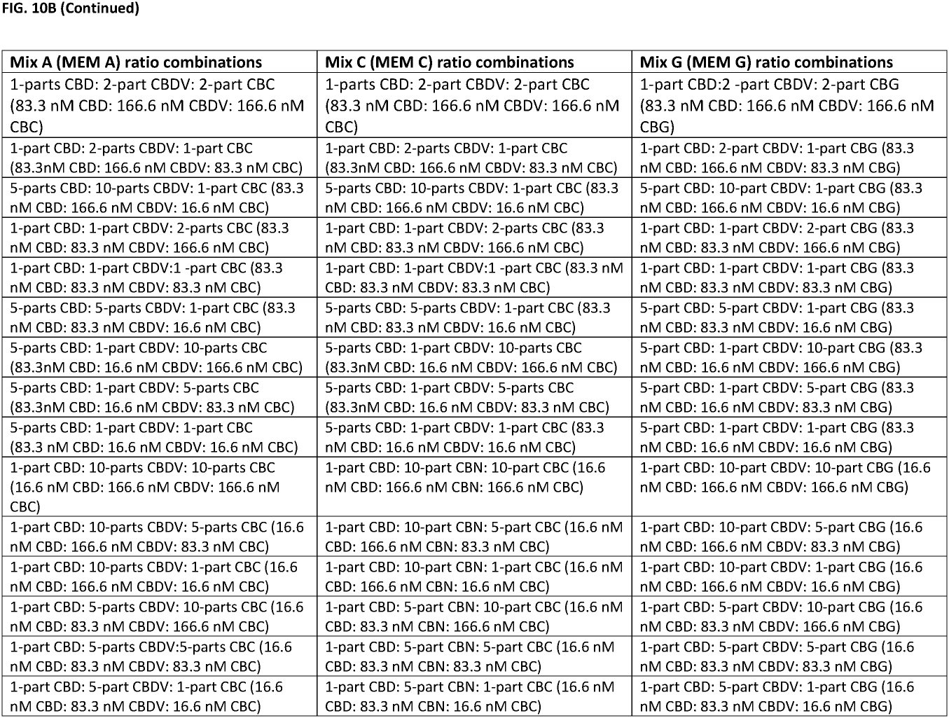

Initial experiments were conducted to determine the effective concentration ranges of each of the five pure, individual cannabinoids. In general, the cannabinoids tested acutely showed that as the concentration was increased, the normal response to a light to dark transition was abolished. The effective concentration range was considered the concentrations between a no observable effect level (NOE) and a level that had a minimal statistically significant effect on behavior (Table 1 of FIG. 9). At the concentrations of the cannabinoids tested there was a slight opposition to the 150 µM OHDA induced hypoactivity, however, the effects were not significant.

In order to assess possible potentiating effects between the cannabinoids, three component, equimolar minimum essential mixtures (E-MEM) of the 5 cannabinoids were prepared (FIG. 4A). Three of these E-MEMs; A, C and G showed a significant opposition to the OHDA induced hypoactivity as measured by the total distance travelled (FIG. 4C) while not displaying any effect on carrier control larvae (FIG. 4B).

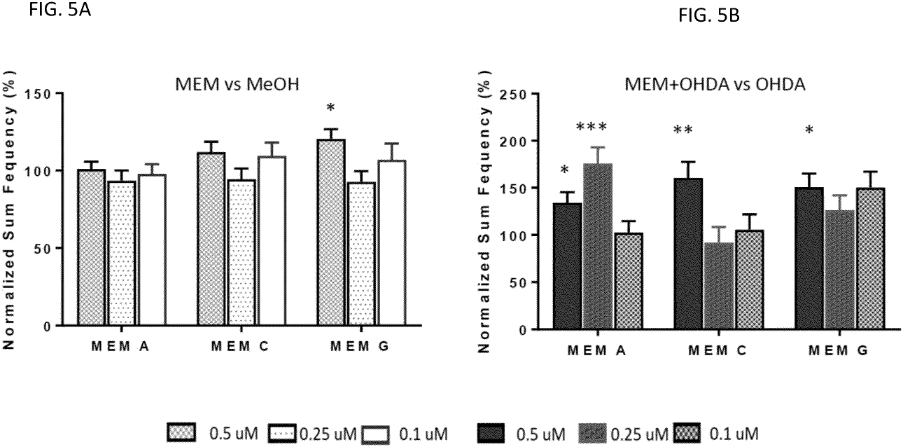

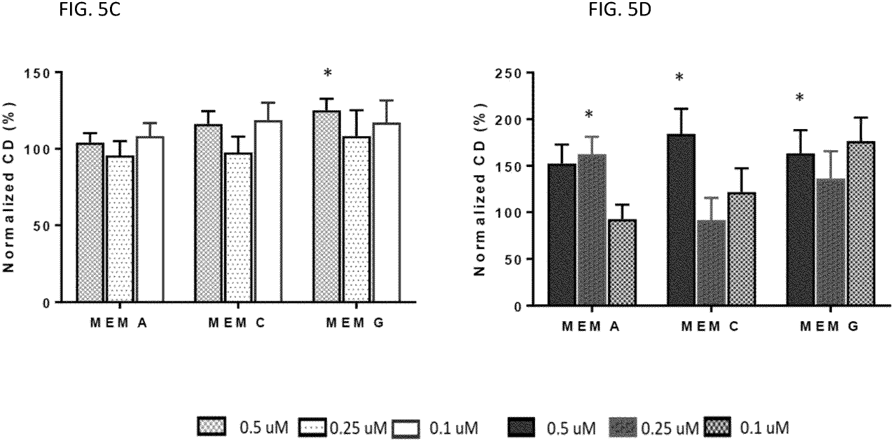

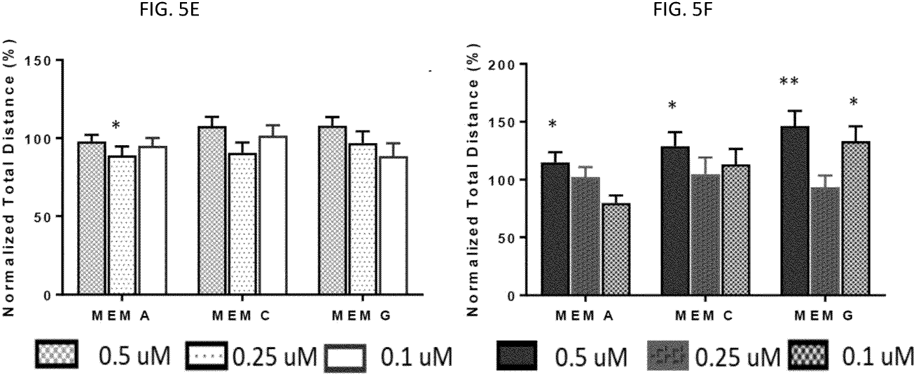

The equimolar MEMs (A, C and G) were further analyzed using the refined activity metrics. At 500 nM equimolar all three MEMs except MEM-C, significantly affected Frequency, Cumulative Duration (CD) and Total distance as measured using activity metrics (FIGS. 5A-5F). 250 nM dilutions of the MEMs abolished these changes, except with relation to Frequency and CD in 250 nM MEM A (FIG. 5B). No recovery was apparent for the 100 nM equimolar MEM experiments for Frequency or CD metrics. 500 nM MEM G increased the activity of MeOH treated control larvae (FIGS. 5A and 5C).

### Identification of Optimal, Defined Cannabinoid-Ratio Minimum Essential Mixture (DCR-MEM)

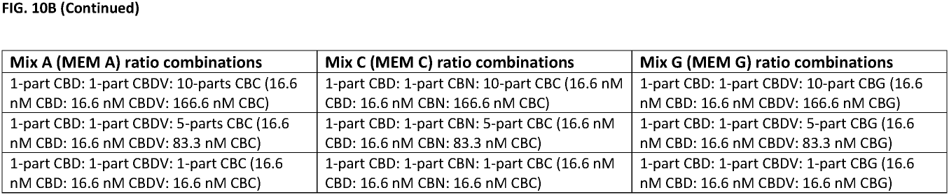

Based on the results from the equimolar mixture experiments, a comprehensive series of experiments were performed where one or two components of each mixture were reduced by 50% (1:2 ratio relative to the equimolar MEM) and/or 90% (1:10 ratio relative to the equimolar MEM) to produce novel molar ratios for further efficacy studies. The defined cannabinoid-ratio minimum essential mixtures (DCRMEMs) that produced the most significant opposition to the PD-like effects of OHDA is described herein (FIG. 6) and a comprehensive summary of all of the results is included in Table 2 and Table 3 of FIGS. 10A-10B. Optimization of all three E-MEMs led to a substantial opposition to PD-like, OHDA mediated changes in Frequency and Cumulative Duration, while having no effect on methanol treated controls (FIG. 6). For E-MEM A, the 250 nM equimolar mixture performed as well as the optimized version (a 50% decrease in CBDV from the original 500 nM dilution). None of the MEM C optimized ratios were able to improve upon the 500 nM original dilution without causing increased activity against methanol controls. Several optimized ratios were developed for MEM G that improved upon the original equimolar response to OHDA, while not affecting methanol treated larvae. Specifically, ratio 5 (mix G: 1 part CBD: 2 parts CBDV: 1 part CBG of FIG. 6) displayed the greatest response in both the Frequency and Cumulative Duration metrics (FIG. 6).

### Summary of Findings

The use of the zebrafish OHDA model to reduce complexity of cannabinoid mixtures demonstrated efficacy in cell-based MPTP-neuroprotection and dopamine release assays.

Because there are hundreds of ingredients in Cannabis extracts that may have therapeutic potential along with an extremely large number of possible combinations of these constituents, it may be impossible to systematically test every combination in order to identify the optimal mixture of purified components that make Cannabis derived compounds good potential therapeutics. Therefore, one approach to identify the essential elements in Cannabis is to logically and experimentally reduce the complexity of these mixtures while retaining as much of the original bioactivity as possible. More specifically, one may increase the probability of a positive therapeutic outcome by identifying the top performing mixture at each level of reduced complexity without exhaustively (and blindly) attempting to test every possible variation.

In this study, the utility of a multitiered approach was demonstrated to identify minimum essential mixtures that are pharmacologically active in cell and animal models of PD. Additionally, because cannabinoids reportedly modulate DA secretion, dopamine-release assays were also used to measure the ability of these compounds to supplement low dopamine production levels through increased DA secretion. The cell-based data demonstrated clearly that the cannabinoids provided both neuroprotective and dopamine secretion abilities not seen in the terpene mixtures alone (FIGS. 1 and 2).

The present inventors surprisingly found that the maximal effects seen in the mixtures represent more than the expected sum of their individual effects. This result provides further support for the ‘entourage hypothesis’, which explains the ability of Cannabis-derived ingredients to act synergistically by enhancing or diminishing the net effectiveness of a therapy cannabinoid pharmacology.

A larval zebrafish model was selected to assess the ability of the cannabinoid mixtures to alleviate the movement disorders associated with Parkinson’s disease. Zebrafish larval locomotion is often divided into burst swimming (high velocity) and slow swimming (lower velocity) states. Not surprisingly, these disparate behaviors have been mapped to multiple different regions of the brain that are enervated substantially by dopaminergic neurons.

In this study, a third activity state was discovered, characterized by a zero-velocity, tremor-like movement. Attempts to quantify this activity led to develop advanced analytics of larval behavior. The use of the zebrafish OHDA model allowed for a reduction in the complexity and a refinement of the ratios of the original cannabinoid mixtures that demonstrated efficacy in the cell-based assays. MEMs containing CBD and CBDV or CBD and CBC demonstrated the greatest therapeutic potential.

### Support for Evaluating the Endocannabinoid System as a Drug Development Target for Parkinson’s Disease

The data in this study supports the idea that endocannabinoid (ECB)-targeting will modify Parkinsonian movement disorders and provides a MEM for further experimentation.

In this work, cell models of PD allowed for the screening of complex mixtures of Cannabis-derived ingredients and identified the cannabinoids as those being responsible for the neuroprotective (MPTP/MPP assays) and dopamine secretory effects. Using the OHDA zebrafish model of Parkinsonian movement disorders, three MEMs containing equal parts of three cannabinoids each were identified that could significantly relieve the OHDA-related motor symptoms. An additional 63 variations using different ratios of the 3 original MEM (MEM A, MEM C, and MEM G) were also tested, and 22 out of 63 of the defined cannabinoid ratio variations also significantly improved OHDA-related symptoms in zebrafish (FIG. 10A). Five of these 22 MEMs (FIG. 6) outperformed the original mixtures and will be further tested in more advanced animal models to develop new therapeutic options for Parkinson’s patients and Parkinsonian movement disorders.

### 7. EQUIVALENTS AND INCORPORATION BY REFERENCE

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

1. A pharmaceutically active ingredient, comprising:
a ratio of:
(a) cannabidiol (CBD),
(b) cannabidivarin (CBDV), and
(c) cannabichromene (CBC).

2. (canceled)

3. The pharmaceutically active ingredient of claim 1, wherein the ratio is a molar ratio selected from:
1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of:
(a) cannabidiol (CBD),
(b) cannabidivarin (CBDV), and
(c) cannabichromene (CBC).

4. The pharmaceutically active ingredient of claim 3, wherein the molar ratio is 2:1:2 of CBD, CBDV, and CBC.

5. A pharmaceutical composition comprising the pharmaceutically active ingredient of claim 1 and a pharmaceutically acceptable carrier or diluent.

6. (canceled)

7. (canceled)

8. (canceled)

9. (canceled)

10. (canceled)

11. (canceled)

12. A unit dosage form comprising the active pharmaceutical ingredient of claim 1.

13. The unit dosage form of claim 12 for a 2:1:2 ratio of CBD:CBDV:CBC, comprising:
i. 0.2 mg to 40 mg of CBD;
ii. 0.1 mg to 20 mg of CBDV;
iii. 0.2 mg to 40 mg of CBC.

14. A method of treating neurodegenerative disease, the method comprising:
administering an effective amount a pharmaceutical composition of claim 4 to a patient having a neurodegenerative disease.

15. The method of claim 13, wherein the neurodegenerative disease is Alzheimer’s disease, Parkinson’s disease, Parkinsonian movement disorder, Lewy Body Dementia, or Huntington’s disease.

16. (canceled)

17. The method of claim 14, wherein the pharmaceutical composition is administered p.r.n or orally.

18. (canceled)

19. The method of claim 17, wherein the pharmaceutical composition is administered as an oral disintegrating tablet (ODT).

20. (canceled)

21. (canceled)

22. (canceled)

23. (canceled)

24. (canceled)

25. A method of treating a dopamine-related Parkinson’s Disease symptom, the method comprising: administering an effective amount of a pharmaceutical composition of claim 4 to a patient having a dopamine-related Parkinson’s Disease symptom.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A pharmaceutically active ingredient, comprising:
a ratio of:
(a) cannabidiol (CBD),
(b) cannabichromene (CBC), and
(c) cannabidiol (CBN).

39. (canceled)

40. The pharmaceutically active ingredient of claim 38,, wherein the ratio is a molar ratio selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of:
(a) cannabidiol (CBD),
(b) cannabichromene (CBC), and
(c) cannabidiol (CBN).

41. The pharmaceutically active ingredient of claim 40, wherein the ratio is 1:1:1 of CBD, CBC, and CBN.

42. A pharmaceutical composition comprising the pharmaceutically active ingredient of claim 38 and a pharmaceutically acceptable carrier or diluent.

43. (canceled)

44. (canceled)

45. (canceled)

46. (canceled)

47. (canceled)

48. (canceled)

49. A unit dosage form comprising the active pharmaceutical ingredient of claim 38.

50. A unit dosage form of claim 49, for a 1:1:1 ratio of CBD:CBDV:CBG, comprising:
i. 0.1 mg to 20 mg of CBD;
ii. 0.1 mg to 20 mg of CBN; and
iii. 0.1 mg to 20 mg of CBC.

51. A method of treating neurodegenerative disease, the method comprising:
administering an effective amount of a pharmaceutical composition of claim 42 to a patient having a neurodegenerative disease.

52. (canceled)

53. (canceled)

54. (canceled)

55. (canceled)

56. (canceled)

57. (canceled)

58. (canceled)

59. (canceled)

60. (canceled)

61. (canceled)

62. A method of treating a dopamine-related Parkinson’s Disease symptom, the method comprising: administering an effective amount of a pharmaceutical composition of claim 51 to a patient having a dopamine-related Parkinson’s Disease symptom.

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. A pharmaceutically active ingredient, comprising:
a ratio of:
(a) cannabidiol (CBD),
(b) cannabidivarin (CBDV), and
(c) cannabigerol (CBG).

75. (canceled)

76. The pharmaceutically active ingredient of claim 74, wherein the ratio is a molar ratio selected from: 1:1:1; 2:2:1; 10:10:1; 2:1:2; 2:1:1; 10:5:1; 10:1:10; 10:1:5; 10:1:1; 1:2:2; 1:2:1; 5:10:1; 1:1:2; 5:5:1; 5:1:10; 5:1:5; 5:1:1; 1:10:10; 1:10:5; 1:10:1; 1:5:10; 1:5:5; 1:5:1; 1:1:10; and 1:1:5 ratio of:
(a) cannabidiol (CBD),
(b) cannabidivarin (CBDV), and
(c) cannabigerol (CBG).

77. The pharmaceutically active ingredient of claim 76, wherein the ratio is 1:2:1 of CBD, CBDV, and CBG.

78. A pharmaceutical composition comprising the pharmaceutically active ingredient of claim 74 and a pharmaceutically acceptable carrier or diluent.

79. (canceled)

80. (canceled)

81. (canceled)

82. (canceled)

83. (canceled)

84. (canceled)

85. A unit dosage form comprising the active pharmaceutical ingredient of claim 74.

86. The unit dosage form of claim 85, for a 1:2:1 ratio of CBD:CBDV:CBG, comprising:
i. 0.1 mg to 20 mg of CBD;
ii. 0.2 mg to 40 mg of CBDV; and
iii. 0.1 mg to 20 mg of CBG.

87. The unit dosage form of claim 85, for a 1:2:10 ratio of CBD:CBDV:CBG
i. 0.01 mg to 5 mg CBD;
ii. 0.02 mg to 10 mg CBDV
iii. 1.0 mg to 50 mg CBG.

88. A method of treating neurodegenerative disease, the method comprising:
administering an effective amount of a pharmaceutical composition of claim 78 to a patient having a neurodegenerative disease.

89. (canceled)

90. (canceled)

91. (canceled)

92. (canceled)

93. (canceled)

94. (canceled)

95. (canceled)

96. (canceled)

97. (canceled)

98. (canceled)

99. A method of treating a dopamine-related Parkinson’s Disease symptom, the method comprising: administering an effective amount of a pharmaceutical composition of claim 78 to a patient having a dopamine-related Parkinson’s Disease symptom.

100. (canceled)

101. (canceled)

102. (canceled)

103. (canceled)

104. (canceled)

105. (canceled)

106. (canceled)

107. (canceled)

108. (canceled)

109. (canceled)

110. (canceled)
